(19) United States
(12) Patent Application Publication
(10) Pub. No.: US 2020/0205773 A1
(43) Pub. Date: Jul. 2, 2020

(54) ULTRASOUND IMAGING SYSTEM

(71) Applicant: UltraDiagnostics, Inc., St. Augustine, FL (US)

(72) Inventors: Kullervo Hynynen, Toronto (CA); Anna Kristoffersen, Pickering (CA); Tyler Portelli, Toronto (CA)

(21) Appl. No.: 16/720,985

(22) Filed: Dec. 19, 2019

Related U.S. Application Data
(60) Provisional application No. 62/786,193, filed on Dec. 28, 2018.

(51) Int. Cl. A61B 8/08; A61B 8/00; A61B 8/14

(52) U.S. Cl. CPC A61B 8/0808; A61B 8/4488; A61B 8/485; A61B 8/0875; A61B 8/4411; A61B 8/4281; A61B 8/5246; A61B 8/5207; A61B 8/4245; A61B 8/5269; A61B 8/483; A61B 8/14; A61B 8/54

(57) ABSTRACT

An ultrasound imaging system for imaging soft tissue through bone matter of a subject. The imaging system transmits ultrasound waves via an ultrasound probe toward the subject's bone material at a plurality of incidence angles so that ultrasound waves may pass through and reflect back through bone as both longitudinal and shear waves, which are all used in combination for imaging. The system includes a switch to connect the transducer elements to a commercially available ultrasound driving system, which allow the imaging system to utilize an ultrasound driving system which has fewer electrical transmit/receive channels than the ultrasound probe. The host controller processes the received ultrasound signals to form an image of the subject's soft tissue through matter. The image reconstruction method, along with tracking information, allows the creation of whole-brain two-dimensional, 2D orthogonal, or three-dimensional images, as well as time lapse four-dimensional or tomographical ultrasound images.

### CLAIM OF PRIORITY

This application claims the benefit of priority under 35 U.S.C. § 119 to U.S. Provisional Patent Application Ser. No. 62/786,193 entitled “Matrix Imaging Mode Ultrasound System Usable for Transcranial Ultrasound Imaging” filed on Dec. 28 2018, which is incorporated herein in its entirety.

### STATEMENT REGARDING FEDERALLY SPONSORED RESEARCH

This invention was made with Government support under contract W81XWH-15-C-0115 entitled “Portable Ultrasound Imaging of the Brain for Use in Forward Battlefield Areas” awarded by the USA MED Research Acquisition Activity of the United States Army (USAMRAA). The Government has certain rights in the invention.

### FIELD OF THE INVENTION

This invention relates to ultrasound imaging systems and, more particularly, to a dual wave ultrasound imaging system.

### BACKGROUND OF THE INVENTION

Outcomes for various brain conditions, including traumatic brain injury (TBI), improve when the care routine includes imaging. Diagnostic imaging practices for conditions of the brain including stroke, hydrocephalus and TBI for example, rely on computed tomography (CT) and magnetic resonance imaging (MRI) imaging, but these imaging modalities are costly, and in the case of CT, are accompanied with risks (e.g. patient exposure to large doses of ionizing radiation), and accordingly repeated imaging over the course of the injury or condition, while medically (diagnostically) desirable, is contra-indicated by such risks for patient safety. Additionally, there is often limited access to CT and MR imaging in field hospitals or rural areas. Ultrasound is non-ionizing and generally regarded as a safe imaging modality. The presence, however, of the skull bone introduces many challenges.

Transcranial ultrasound imaging has been limited to temporal or sub-occipital acoustic windows, where typical imaging frequencies can be used. Alternative acoustic windows suffer from variability of skull morphology and thicknesses. Improvements can be made using multiple transducers to simultaneously image through both temporal windows allowing a three-dimensional image of the Circle of Willis. Since the mid 20th century, transcranial Doppler (TCD) has been used to non-invasively measure arterial cerebral blood flow velocities and is widely used to diagnose stroke and to assess recanalization. However, the field of view available for this technique is limited, and imaging artifacts caused by the skull bone are present. Development of three-dimensional (3D) ultrasound imaging has improved vessel imaging, but traditional ultrasound imaging techniques cannot access most areas of the brain, and skull thickness completely confounds ultrasound imaging for a significant number of patients.

With traditional ultrasound imaging, the large acoustic mismatch between skull bone and surrounding tissue results in acoustic power losses between approximately 30% and 80% at “normal incidence” (when the direction of transmission of ultrasound waves is perpendicular to the plane of the bone surface—90 degrees), due to strong reflections. Signals from echoes within the brain structure are weak compared to reflections from bone-tissue interfaces, which also confounds effective imaging. The skull bone is also highly attenuating, further reducing the signals returning from structures below the skull bone. Distortion of the transmitted ultrasound as it passes through the skull results in artifacts and beamforming challenges. These distortion effects worsen as the ultrasound wavelength size approaches the same order of magnitude as the phase shifts introduced by the skull. Adaptive beamforming techniques have been proposed to combat these challenges. Another approach proposed has been to aid focusing with signals from bubble-emissions arising from focusing the transmitted beam through the skull and into the region of interest to be imaged. Previous tests associated with U.S. Pat. No. 7,175,599, have demonstrated single-element A-Mode trans-skull detection of sinus opacification and detection of ventricle boundaries through the skull bone, with the use of shear waves. The invention of this patent also required the use of a separate mechanical positioning device to target a linear region along a line of transmission of the ultrasound main beam. However, this scheme has not proven effective in generating clinically useful images. Other disadvantages may exist.

### SUMMARY OF THE INVENTION

A dual wave ultrasound imaging system of the present invention for imaging soft tissue through bone matter of a patient/subject, that transmits longitudinal ultrasound waves via an ultrasound probe such that the ultrasound waves are delivered toward the patient/subject's bone material, such as the skull of a patient/subject, at a plurality of “incident angles” (the direction of propagation of the ultrasound waves in relation to the normal to the bone surface) ranging from a “normal” incidence angle (when the direction of transmission of the ultrasound waves is perpendicular to the bone layer), through a series of incident angles that are both less than, greater than and equal to the longitudinal critical angle (defined as an angle between the normal to the bone layer and the direction of transmission of the ultrasound, above which no longitudinal waves are propagated through the bone layer). When a longitudinal ultrasound wave is transmitted at an incident angle between 30° and 60° from normal to the plane of the bone surface, the longitudinal wave is converted to shear waves that propagate through the bone and then are that converted back to longitudinal waves as they exit the interior surface of the bone. There is evidence that when longitudinal waves are transmitted at an incident angle between ˜25 and ˜30 there ultrasound propagates through the bone as both longitudinal and shear waves. The ultrasound probe of the present disclosure is configured to ensure that transmitted ultrasound waves will propagate through the bone either as shear waves or as longitudinal waves depending on the angle of incidence to the bone, to then be scattered or reflected back from soft tissue anatomy to propagate back again through the bone layer, both as shear waves and as longitudinal waves.

Although longitudinal waves suffer from significant attenuation and distortion while propagating through bone, they have the advantage of reflecting a stronger signal than shear waves, and accordingly; rather than attempting to inhibit mechanically or electronically the transmission of longitudinal ultrasound waves that may be propagating at an angle to the normal of the bone layer that is lower than the longitudinal wave critical angle, the present disclosure intentionally transmits longitudinal ultrasound waves into the skull of the patient at a plurality of angles including 0-˜60 degrees from normal to the plane of the surface of the bone, and applies imaging algorithms to correct longitudinal wave distortions, and receives both longitudinal waves and converted shear waves that have been reflected or backscattered from features on the interior of the skull bone, and makes use of all reflected waves, both converted shear waves and longitudinal waves in image reconstruction. The present disclosure does not require the use of a separate mechanical positioning device for transmission of longitudinal ultrasound waves at various angles to the normal of a bone layer, and allows free movement of the hand-held transducer probe by the sonographer to enable a larger field of view in order to produce whole-brain two-dimensional (2D) images, 2D orthogonal images, or three-dimensional (3D) images, as well as time lapse four-dimensional (4D) images of either orthogonal 2D ultrasound images, 3D ultrasound images, or tomographical ultrasound images.

The dual wave ultrasound imaging system is designed to create and employ four different transmit/receive combinations uniquely for trans-bone imaging: 1) Zero Wave Conversion—Transmission of longitudinal waves at such an angle that propagate first through the bone as longitudinal waves which then reflect and propagate back through the bone as longitudinal waves, to be received by the transducer as reflected longitudinal waves (FIG. 29); 2) Transmit (Tx) Double Wave Conversion—Transmission of longitudinal waves at such an angle that they propagate through the bone as shear waves, which convert to longitudinal waves upon exiting the interior surface of the bone, which then reflect back at such angle that they propagate through and exit the exterior surface of the bone as longitudinal waves) to be received by the transducer as longitudinal waves (FIG. 30); 3) Receive (Rx) Double Wave Conversion—Transmission of longitudinal waves at such an angle that they propagate through the bone as longitudinal waves which then reflect back at such an angle that they propagate back through the bone as shear waves, which then convert back from shear waves to longitudinal waves upon exiting the exterior surface of the bone to be received by the transducer as longitudinal waves (FIG. 31); and 4) Quadruple Wave Conversion—Transmission of longitudinal waves at such an angle that they propagate through the bone as shear waves, which then exit the internal surface of the bone and convert to longitudinal waves, which then reflect back at such an angle that they propagate back through the bone as shear waves, and then convert back again to longitudinal waves as they exit the exterior surface of the bone to be received by the transducer as longitudinal waves (FIG. 32).

Since reflected and backscattered ultrasound may be incident upon the bone layer at many angles, in order to discriminate between the modes of propagation of the ultrasound through the bone, a “synthetic receive aperture” is employed. The synthetic receive aperture is a processing algorithm which is used to control which transducer elements contribute to the image reconstruction. An algorithm is used to determine the angle of incidence upon the bone layer of the reflected ultrasound waves that may be originating from each pixel or voxel. This is used to control which elements contribute to the reconstruction of each pixel or voxel based on the mode of propagation of ultrasound through the bone (i.e. longitudinal or shear mode) originating from that voxel.

Such received longitudinal waves and converted shear waves are received by the transducer and converted to electronic signals (hereby referred to as “received radio frequency (RF) signals”). Such received RF signals are then digitized by an ultrasound driving system as “digitized received RF signals” (IQ data or other data formats may be equivalently used), where the digitized, received RF signals from all reflected waves are passed to the host controller and used in an image reconstruction algorithm to beamform the received RF signals onto a grid of pixels (or voxels) to produce an ultrasound image in conjunction with tracking of the ultrasound probe to co-register the ultrasound imaging pixels (or voxels) from each frame (or transducer position); for the purpose of creating a two-dimensional or three-dimensional image of a subject's soft tissue through bone material of the subject/patient.

In one disclosed embodiment, for the purpose of diagnostic imaging, an ultrasound transducer probe, with at least one transmit/receive segment or pad including an array of transducer elements (such as piezo electric crystals), transmits and receives ultrasound waves in the four transmit/receive combinations explained herein. A full or sparse random receive transducer element array is positioned around the transmit/receive pads to maximize detection of waves from reflected and scattered features below the skull bone, while keeping the footprint size of the element array feasible for a handheld device. The hand-held imaging probe is preferably configured to optimize central frequency, bandwidth, element layout, and array geometry for trans-bone applications. Reflections of the transmitted ultrasound beam from the outer and inner surfaces of the skull are used to predict and filter out artifacts associated with multiple reflections in order to distinguish between artifacts from the bone layer and reflections from tissue. A digital map of the individual characteristics (internal and external surfaces) of each skull is created and used to estimate and correct for the delay introduced by the bone layer, in order to correct for aberration artifacts. Phase and amplitude corrections to the received longitudinal waves (including converted shear waves) are used in receive beamforming to correct for bone aberrations.

The design of the ultrasound transducer probe element array, including transmit/receive pads and receive pads, allows multi-beam transmission (transmission from multiple pads within the transducer) of longitudinal ultrasound waves at a variety of steering (incident) angles, up to the shear wave critical angle, without requiring a separate positioning device to enable transmission at the desired incident angle. Additionally, or alternatively, the sonographer can achieve greater or different fields of view manually (i.e., by manually repositioning the ultrasound transducer probe 68 on the subject's or patient's head).

Specific embodiments are described herein with reference to an ultrasound probe and related ultrasound system switch and processing systems and software, and image reconstruction software and tracking devices intended specifically for transcranial (trans-skull) dual wave imaging applications. The software referred to in this document was developed for the purpose of managing the various transmit/receive combination of the dual wave ultrasound imaging system described in paragraph 9 above, and generating dual wave images, combining the digitized received RF signals (received from longitudinal ultrasound waves and may be reconstructed from IQ data) from the transducer elements for both converted shear waves and longitudinal waves. The same or similar ultrasound probes and related ultrasound system switches and processing systems may be configured for other applications such as, without limitation, ultrasound imaging through other bones and structures such as the sternum (e.g., for imaging of the heart or esophagus), ribs, hip, pelvis, etc.

The dual wave ultrasound imaging system of the present invention enables repeated monitoring and reassessment of brain injuries (or other conditions) after initial triage and treatment, without the need for repeated CT or MM studies, which may be impractical due to such factors as patient stability, convenient access to equipment and technicians, or issues/concerns relating to repeated exposure to large amounts of ionizing radiation. Other advantages include small size, portability and dramatically lower cost compared to other technologies such as CT or MRI.

One embodiment of the present disclosure is an ultrasound imaging system comprising an ultrasound transducer probe includes a face configured to contact a subject, the face including an array of transducer elements, the array of transducer elements includes at least one first transmit pad that includes at least one first active transducer element, at least one second transmit pad that includes at least one second active transducer element, and at least one receive pad, wherein the at least one first active transducer element is capable of transmitting longitudinal ultrasound waves at a first incident angle with respect to a bone of the subject so that waves may propagate through the bone as shear waves and wherein the at least one second active transducer element is capable of transmitting longitudinal ultrasound waves at a second incident angle with respect to the bone so that the waves may propagate through the bone as longitudinal waves. The ultrasound imaging system includes a host controller, an ultrasound driving system, and an ultrasound transducer probe. The ultrasound imaging system includes an ultrasound system switch that connects the ultrasound driving system to the ultrasound transducer probe, wherein the host controller controls operation of the ultrasound transducer probe via the ultrasound driving system. The host controller of the ultrasound imaging system commands the ultrasound driving system to generate radio frequency (RF) signals that are used by the transducer probe to generate ultrasound waves. Upon receipt of commands from host controller, the ultrasound driving system causes the ultrasound transducer probe to generate ultrasound waves at the first incident angle and at the second incident angle. The ultrasound driving system captures electronic signals produced by ultrasound waves received by the at least one receive pad of the ultrasound transducer probe via the ultrasound system switch and digitizes the received electronic signals. The host controller forms an image of the subject based on the digitized received electronic signals.

One embodiment of the present disclosure is an ultrasound transducer probe. The ultrasound transducer probe comprises a face configured to contact a subject. The ultrasound transducer probe includes an array of transducer elements including at least one first transmit pad and at least one second transmit pad, the first transmit pad includes at least one first active transducer element and the at least one second pad includes at least one second active transducer element, and at least one receive pad. The at least one first active transducer element is capable of delivering longitudinal ultrasound waves at a first incident angle with respect to a bone of the subject that produces shear waves through the bone. The at least one second active transducer element is capable of delivering longitudinal ultrasound waves at a second incident angle with respect to the bone of the subject so that it produces longitudinal waves though the bone.

One embodiment of the present disclosure is an ultrasound imaging method. The ultrasound imaging method comprises transmitting longitudinal ultrasound waves via an ultrasound probe toward a target at a plurality of incident angles, wherein at least a first incident angle is below a longitudinal wave critical angle and wherein at a second incident angles is above the longitudinal wave critical angle and below a shear wave critical angle. The method includes receiving reflected longitudinal ultrasound waves via the ultrasound probe. The method includes producing received radio frequency (RF) signals via the ultrasound probe based on the received reflected longitudinal ultrasound waves. The method includes receiving backscattered longitudinal ultrasound waves via the ultrasound probe. The method includes producing received RF signals via the ultrasound probe based on the received backscattered longitudinal ultrasound waves. The method includes digitizing the received RF signals to form digitized RF signals. The method includes processing the digitized RF signals to form an image of the target.

The target may be soft tissue, and the incident angles are with respect to normal to the plane of a bone layer, and the longitudinal ultrasound waves are transmitted through the bone layer. The first incident angle may enable longitudinal waves to pass through the bone and the second incident angle may enable a quadruple conversion of the longitudinal waves within the bone. The transmission of longitudinal ultrasound waves may include transmission of longitudinal ultrasound waves such that the longitudinal ultrasound waves propagate through the bone layer as longitudinal waves which then reflect and propagate back through the bone layer as longitudinal waves to be received by the transducer as reflected longitudinal waves. The transmission of longitudinal ultrasound waves may include transmission of longitudinal ultrasound waves such that the longitudinal ultrasound waves propagate through the bone layer as shear waves, which convert to longitudinal waves upon exiting the bone layer, which then reflect back at such angle that the reflected waves propagate through and exit the bone layer as longitudinal waves to be received by the transducer as longitudinal waves. The transmission of longitudinal ultrasound waves may include transmission of longitudinal ultrasound waves such that the longitudinal ultrasound waves propagate through the bone layer as longitudinal waves which then reflect back at such angle that the reflected waves propagate through the bone layer as shear waves, which then convert from shear waves to longitudinal waves upon exiting the bone layer to be received by the transducer as longitudinal waves. The transmission of longitudinal ultrasound waves may include transmission of longitudinal ultrasound waves such that the longitudinal ultrasound waves propagate through the bone as shear waves, which then exit the bone layer and convert to longitudinal waves, which then reflect back at such an angle that they propagate back through the bone layer as shear waves, and then convert back again to longitudinal waves as they exit the bone layer to be received by the transducer as longitudinal waves.

Further objects, features and advantages will become apparent upon consideration of the following detailed description when taken in conjunction with the drawings and the appended claims.

### BRIEF DESCRIPTION OF THE DRAWINGS

FIG. 1A is a schematic diagram showing longitudinal ultrasound waves delivered to the skull of patient at an angle with respect to normal, which angle is less than the longitudinal wave critical angle.

FIG. 1B is a schematic diagram showing longitudinal ultrasound waves delivered to the skull of patient at an angle with respect to normal, which angle is greater than the longitudinal wave critical angle.

FIG. 2 is a block diagram of an embodiment of a dual wave ultrasound imaging system.

FIGS. 3A and 3B are schematic diagrams of an embodiment of a transducer element array of an ultrasound transducer probe that may be used in connection with a dual wave ultrasound imaging system.

FIG. 4 is an azimuth view of the ultrasound transducer probe used in connection with the dual wave ultrasound imaging system.

FIG. 5 is a schematic diagram of an embodiment of a dual wave ultrasound imaging system including a fast ultrasound system switch for switching between transmit and receive transducer elements of pads of the ultrasound transducer probe.

FIG. 6A is a timing diagram for an ultrasound system switch for the first transmit/receive pad.

FIG. 6B is a timing diagram for an ultrasound system switch for the second transmit/receive pad.

FIG. 7 is a schematic diagram of an embodiment of a dual wave ultrasound imaging system including a fast ultrasound system switch for receive transducer elements of pads of the ultrasound transducer probe.

FIG. 8 is a block diagram showing an embodiment of the organization of the fast ultrasound system switch for receive only pads of the ultrasound transducer probe.

FIG. 9 is a block diagram showing an embodiment of an imaging sequence for one frame of imaging data.

FIG. 10A is a block diagram showing an embodiment of a transmit/receive imaging sequence for one transmit pad.

FIG. 10B is a block diagram showing an embodiment of the transmit imaging sequence for one frame with both an incident angle that is below the longitudinal wave critical angle and a transmit incident angle greater than the longitudinal wave critical angle and below the shear wave critical angle.

FIG. 11 is a block diagram of an embodiment illustrating the processing of the dual wave ultrasound digitized received RF signals.

FIG. 12 is a block diagram of an embodiment of the dual wave ultrasound imaging system that further includes an ultrasound transducer probe optical tracking system.

FIG. 13 is an image illustrating an embodiment of a setup of the transducer optical tracking system.

FIGS. 14A and 14B are images of an embodiment showing a time sequence of two dual wave ultrasound images that illustrate the expansion of a simulated blood ventricle hemorrhage beneath a skull cap using the dual wave ultrasound imaging system.

FIG. 15A is an image of an embodiment of the x-y plane of a simulation medium using the measured density of a skull cap with simulated shrapnel beneath the skull cap.

FIG. 15B is an image of an embodiment of the x-z plane of a simulation medium using the measured density of a skull cap with simulated shrapnel beneath the skull cap.

FIG. 16A is a graph illustrating an embodiment of the contrast performance of square and rectangular piezo electric crystals (elements) as a function of the receiver element density in the ultrasound transducer probe.

FIG. 16B is a graph illustrating an embodiment of the signal-to-noise ratio performance of square and rectangular piezo electric crystals (elements) as a function of the receiver element density in the ultrasound transducer probe.

FIG. 17A is a graph illustrating an embodiment of the signal-to-noise ratio (SNR) performance of various size rectangular piezo electric crystals (elements) as a function of the receiver element density in the ultrasound transducer probe at various frequencies.

FIG. 17B is a graph illustrating an embodiment of the peak signal-to-noise ratio performance of various size rectangular piezo electric crystals (elements) as a function of the receiver element density in the ultrasound transducer probe at various frequencies.

FIG. 17C is a graph illustrating an embodiment of the contrast performance of various size rectangular piezo electric crystals (elements) as a function of the receiver element density in the ultrasound transducer probe at various frequencies.

FIG. 17D is a graph illustrating an embodiment of the contrast-to-noise ratio performance of various size rectangular piezo electric crystals (elements) as a function of the receiver element density in the ultrasound transducer probe at various frequencies.

FIG. 18A is an image of an embodiment of a simulation medium including a skull cap, a midline, and a simulated 5 mm thick (3 cc volume) intraparenchymal hemorrhage.

FIG. 18B is an ultrasound image of an embodiment of the simulation medium of FIG. 18A using an ultrasound transducer probe with a centrally positioned transmit/receive pad (C6) of FIGS. 3A and 3B of the dual wave ultrasound imaging system.

FIG. 18C is an embodiment of a dual wave ultrasound image of the simulation medium of FIG. 18A using an ultrasound transducer probe with an offset transmit/receive pad (C1) of FIGS. 3A and 3B of the dual wave ultrasound imaging system.

FIG. 19 is an image of an embodiment of a simulation medium including a skull cap plus a simulated 17 mm subdural hematoma.

FIG. 20A is an image of an embodiment of a simulation medium including a skull cap plus a midline.

FIG. 20B is a dual wave ultrasound image of an embodiment of the simulation medium of FIG. 20A using the ultrasound transducer probe of FIGS. 3A and 3B with aberration correction to the simulated received RF signals to correct the midline to its expected position.

FIG. 21A shows an embodiment of a phantom skull with a traumatic brain injury for use in connection with the dual wave ultrasound imaging system.

FIG. 21B is in ultrasound image of an embodiment of the phantom skull of FIG. 21A.

FIG. 21C is a single frame of an embodiment of an ultrasound image of the phantom skull with a simulated traumatic brain injury hemorrhage.

FIG. 22A is an embodiment of a slice of in ultrasound image of a skull cap and brain with a simulated subdural hematoma.

FIG. 22B is an embodiment of a three-dimensional image of the slice of FIG. 22A.

FIGS. 23A and 23B are embodiment of ultrasound images of a simulated subdural hematoma below a skull cap.

FIG. 24A is an illustration of an embodiment of a traumatic brain injury phantom showing the location of a central slice of a simulated traumatic brain injury including a pancake shaped balloon and a 7 mm tube filled with blood mimicking fluid.

FIG. 24B is an embodiment of a three-dimensional ultrasound image of one frame of the image shown in FIG. 24A.

FIGS. 24C-24E show the image of FIG. 24B in the x-y, x-z, and z-y planes.

FIGS. 25A-25C are embodiments of three-dimensional images of a skull cap and brain phantom with simulated shrapnel comparing the results of a CT scan and an ultrasound scan.

FIG. 25D is an embodiment of a two-dimensional image of the images shown in FIG. 25C with aberration correction and filtering to remove skull artifacts.

FIGS. 26A-26E are embodiments of three-dimensional images of a skull cap and brain phantom with simulated shrapnel comparing the results of a CT scan and an ultrasound scan.

FIG. 27 is an embodiment of a three-dimensional image of the skull cap with a traumatic brain injury phantom showing a hemorrhage and comparing a CT scan and in ultrasound scan.

FIG. 28A is an embodiment of a graph showing the correlation between results of the CT scan and the ultrasound scan of the present invention where the graph shows the percentage of spheres (shrapnel) visible in each frame for which the spheres also appear in the CT scans.

FIG. 28B is an embodiment of a graph showing the residuals of the linear regression of the location data of the spheres (shrapnel) from FIG. 28A.

FIG. 28C is an embodiment of a graph showing the agreement between the locations of the spheres (shrapnel) located by CT scans and the ultrasound scan.

FIG. 29 is a schematic showing one embodiment of a transmission angle between a transducer and a target.

FIG. 30 is a schematic showing one embodiment of a transmission angle between a transducer and a target.

FIG. 31 is a schematic showing one embodiment of a transmission angle between a transducer and a target.

FIG. 32 is a schematic showing one embodiment of a transmission angle between a transducer and a target.

FIG. 33 is a schematic showing one embodiment of a transmission angle between a transducer and target.

The foregoing figures and the elements depicted therein are not necessarily drawn to consistent scale or to any scale. Unless the context otherwise suggests, like elements are indicated by like numerals. While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the scope of the disclosure as defined by the appended claims.

### DETAILED DESCRIPTION

As used herein, normal incidence means when the direction of transmission of ultrasound waves is perpendicular to the plane of the bone surface, the incidence angle equals zero degrees (“normal”). As used herein, incident angle means the angle between the “normal” to the surface of the bone (zero degrees) and the direction of transmission of the ultrasound wave. For example: ultrasound waves transmitted at a 60 degree angle to the plane of the bone surface, are measured to be at 30 degrees from “normal.” As used herein, longitudinal critical angle means an angle between the normal to the plane of the bone surface and the direction of transmission of the ultrasound, above which no longitudinal waves are propagated through the bone layer (˜30 degrees from normal). As used herein, shear critical angle means an angle above which no shear waves are propagated through the bone (approximately 60 degrees from normal). As used herein, transmit radio frequency (RF) signals means the RF signals generated by the ultrasound driving system and to be applied to the transducer elements to generate ultrasound waves. As used herein, received RF signals means RF signals generated by the transducer elements when they receive ultrasound waves. As used herein, digitized received RF signals means received RF signals from the transducer elements which are then received by an ultrasound transceiver which performs analog and signal digital processing to produce digitized RF signals. As used herein, ultrasound driving system means an ultrasound transceiver for producing RF signals to be applied to transducers, and for receiving RF signals from transducers to produce digitized received RF signals. Also known as ultrasound transceiver.

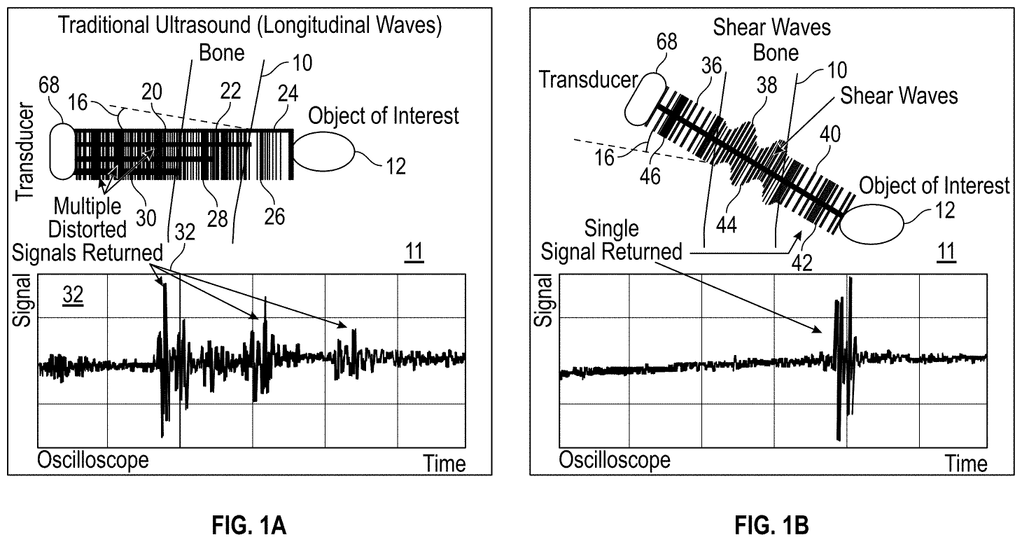

FIG. 1A schematically illustrates a longitudinal ultrasound wave 20 transmitted by an ultrasound transducer probe 68 at an essentially normal angle of incidence (incident angle ˜0°) 16 to normal to the plane of the surface of the bone 10 with soft tissue 11 to the right. FIG. 1B illustrates the longitudinal ultrasound wave 20 transmitted by the ultrasound transducer probe 68 at an angle of incidence 16 of between 25°-60° from the normal angle to the plane of the surface of the bone 10 with soft tissue 11 to the right.

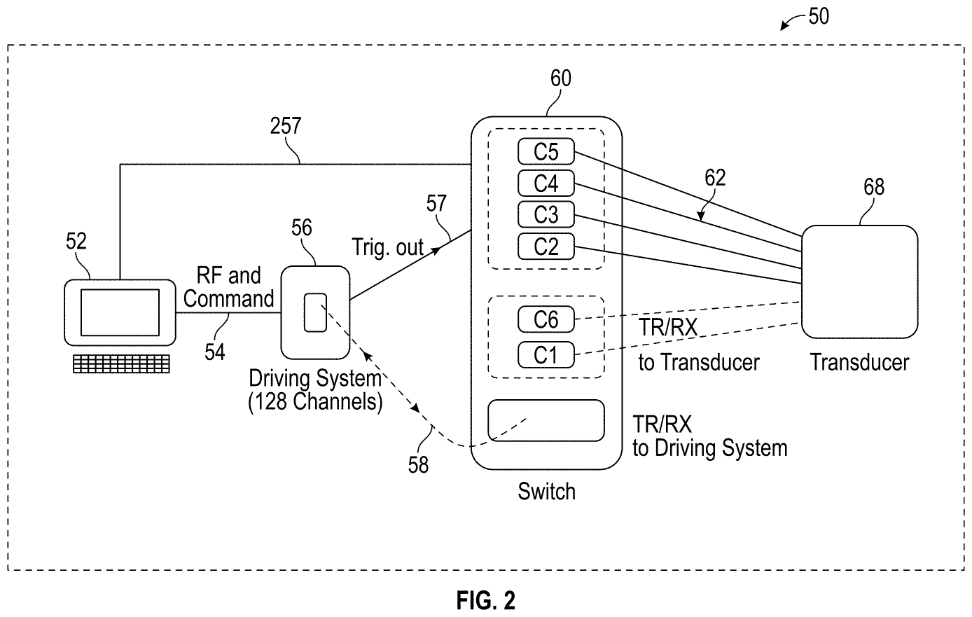

With respect to FIG. 1A, the ultrasound transducer probe 68 of a dual wave ultrasound imaging system 50 (shown in FIG. 2) transmits a longitudinal ultrasound wave 20. The longitudinal ultrasound wave 20 strikes the bone (skull) 10 of a patient at an incident angle 16 of less than about 25° from the normal angle to the bone 10. Because the incident angle 16 is less than 30° from normal, the longitudinal wave 20 is propagated through the bone 10 as a transmitted longitudinal wave 22. The longitudinal wave 22 exits the bone 10 and passes into the soft tissue 11 as a transmitted longitudinal wave 24. Upon encountering the object of interest 12, the transmitted longitudinal wave 24 is reflected as longitudinal wave 26. The reflected longitudinal wave 26 encounters the bone 10 at an incident angle 16 less than the longitudinal wave critical angle and propagates through the bone 10 as a reflected longitudinal wave 28. Upon exiting the bone 20, the reflected longitudinal wave 30 includes multiple reflections (artifacts) of the ultrasound wave due to the large acoustic mismatch between skull bone and surrounding tissue, which can be seen in the digitized received radio frequency (RF) signal 32.

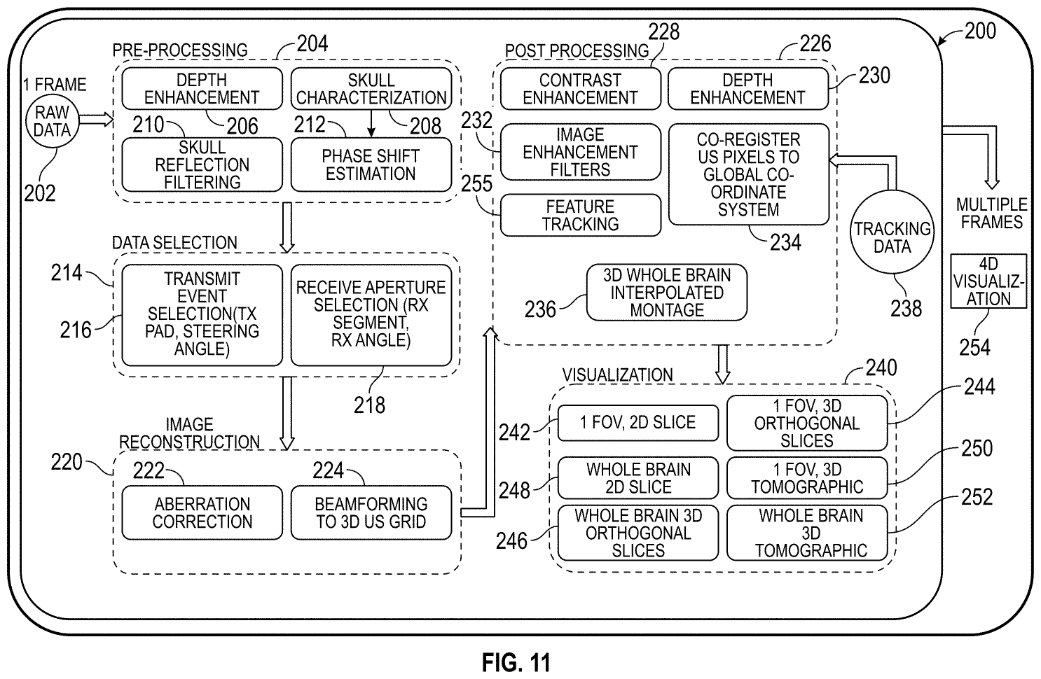

With respect to FIG. 1B, the ultrasound transducer probe 68 of the dual wave ultrasound imaging system 50 transmits longitudinal ultrasound wave 36 via the ultrasound transducer 68. The longitudinal wave 36 strikes the bone (skull) 10 of a patient at an incident angle 16 of between about 30° and 60° from the normal angle to the bone 10. Because the incident angle 16 is greater than 30° from normal and less than 60° from normal, part of the longitudinal wave 36 is reflected and part is transmitted through the bone 10 as a converted shear wave 38. The sheer wave 38 exits the bone 10 and passes into the soft tissue 11 as a longitudinal wave 40. Upon encountering the object of interest 12, the longitudinal wave 40 is reflected as longitudinal wave 42. The reflected longitudinal wave 42 encounters the bone 10 at an incident angle 16 of greater than 30° and less than 60° from normal and propagates through the bone 10 as a converted shear wave 44. Upon exiting the bone 20, the shear wave 44 is converted back to a longitudinal wave 46 which is received by the transducer 68 and converted to a digitized received RF signal (or IQ data) for image processing 200 (shown in FIG. 11).

When a longitudinal ultrasound wave 36 is transmitted at an incident angle between 30° and 60° to the skull bone 10, the longitudinal wave 36 is converted to shear waves 38 that propagate through the bone 10 that are converted back to longitudinal waves 40 at the skull/soft tissue interface on the inside of the skull 10. The propagation of shear waves 38 through the skull bone 10 is refracted and distorted less than longitudinal waves 22, thus improving transcranial imaging. One embodiment of this disclosure takes advantage of four transmit/receive combinations of longitudinal and shear waves as described herein, to allow for volumetric imaging of features 12 below the skull 10 for arbitrary probe positions, specifically by transmitting only in a longitudinal mode and receiving both longitudinal and converted shear waves in a longitudinal mode. By utilizing the various combinations of converted shear waves as well as unconverted longitudinal waves that have been corrected for aberration during receive beamforming, the image quality can be improved for clinical efficacy. The system also utilizes a mode where the angle of incidence is between 25 and 30 degrees, where the ultrasound waves propagate as a mixture of shear mode and longitudinal mode waves.

The illustrative embodiments of the present disclosure transmit longitudinal waves that, depending on incident angle, propagate through the skull as either shear or longitudinal waves into the soft tissue and are reflected back from features to be imaged as longitudinal waves that propagate back through the skull bone 10 either as shear waves or as longitudinal waves.

### Dual Wave Ultrasound Imaging System

Several factors are considered when assessing the design of a dual wave ultrasound imaging system 50 (FIG. 2). Higher frequencies traditionally improve resolution, however, attenuation of both the longitudinal and shear waves increases with frequency when transmitted through bone. Transmitted acoustic power decreases dramatically in shear wave mode. For example, at 840 kHz, the transmitted power is: shear: 6%, longitudinal: 25% and at 548 kHz: shear: 10%, longitudinal: ˜29%. Additionally, a large receive array footprint is desirable in order to capture the reflected ultrasound that is propagating through the bone using shear mode (e.g., incident angles between ˜30° and ˜60°).

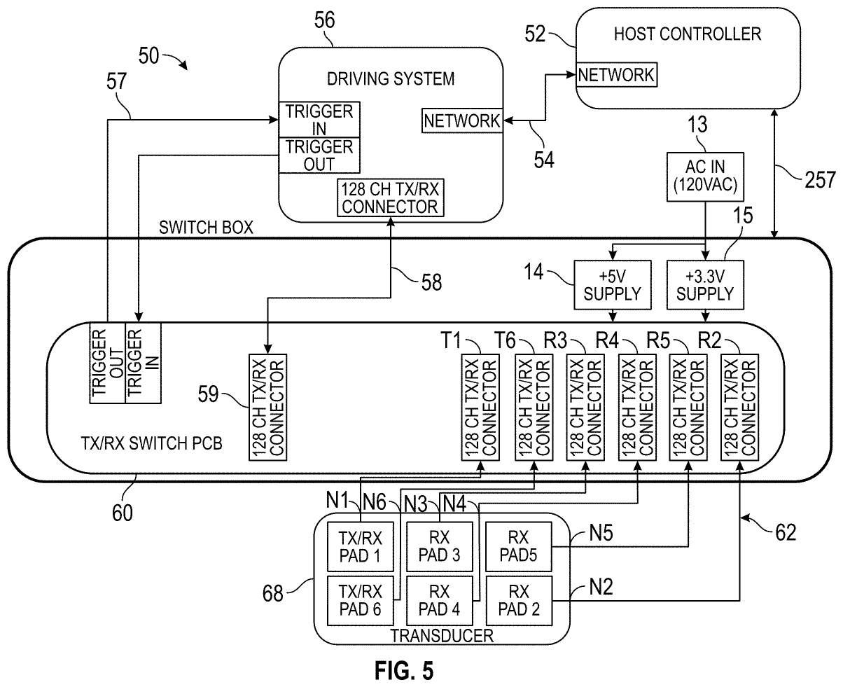

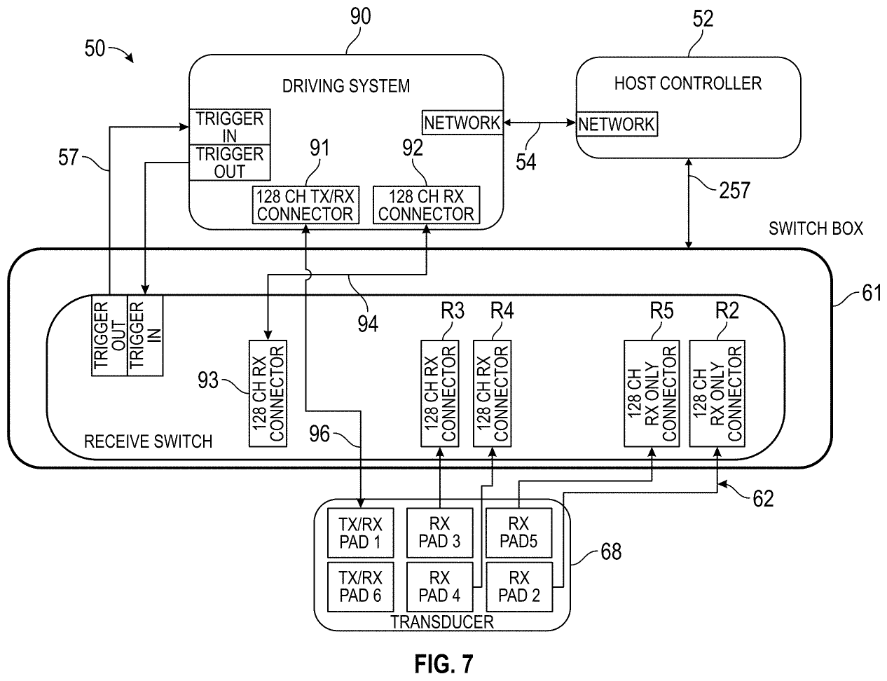

Turning to FIGS. 2, 5, and 7, the dual wave ultrasound imaging system 50 includes a host controller 52, an ultrasound driving system (also known as an ultrasound transceiver) 56, a fast ultrasound system switch 60 (shown in FIG. 2) or 61 (shown in FIG. 7), and an ultrasound transducer probe 68. In FIG. 5, the ultrasound system switch 60 is implemented for transmitting RF signals to and from the ultrasound transducer probe 68. In the embodiment of FIG. 7, the switch 61 is configured as a receive only ultrasound system switch.

The host controller 52 provides command-and-control signals to the driving system 56 via connection 54 that may be a wired or wireless network connection. In turn, the driving system 56 produces a transmit RF signal which is connected via the two-way connection to cable 58 which in turn is connected to the ultrasound system switch 60. The electrical connections to the ultrasound system switch 60 are established to the transducer elements of transducer 68 via cables 62. The ultrasound system switch 60 selects which transmit pad (C1 or C6) is connected to the driving system 56 during transmission of the transmit RF signal from the driving system 56 to the transducer 68. The transducer 68 then converts the transmit RF signals into ultrasound waves. The ultrasound waves are transmitted through a coupling gel pad into the anatomy to be imaged. The returning ultrasound echoes are received by the elements of transducer 68 and converted to a received RF signal which is passed via cables 62 to ultrasound system switch 60, and then passed through cable 58 to driving system 56 or via cable 62 to ultrasound system switch 61 which is in turn passed via cable 94 to driving system 90 via the connector 92. The reflected and scattered ultrasound waves received by each of the active transducer elements (i.e. piezo electric crystals) of the ultrasound transducer probe 68 are converted to received RF signals which are routed to the ultrasound system switch 60 or 61 which then selects which received RF signals from the active transducer elements will be routed to the driving system 56 or 90. The driving system 56 or 90 also provides a trigger signal via line 57. Particularly, the ultrasound system switch 60 routes the transmit RF signals to the active transducer transmit elements of the ultrasound transducer probe 68 which generate longitudinal ultrasound waves. In another embodiment, the driving system 90 produces a transmit RF signal which is connected to transmit pad C1 or C2 of transducer 68 through the two-way connection 91 and via cable 96, and the received RF signals are transmitted to driving system 90 via ultrasound system switch 61.

### Host Controller

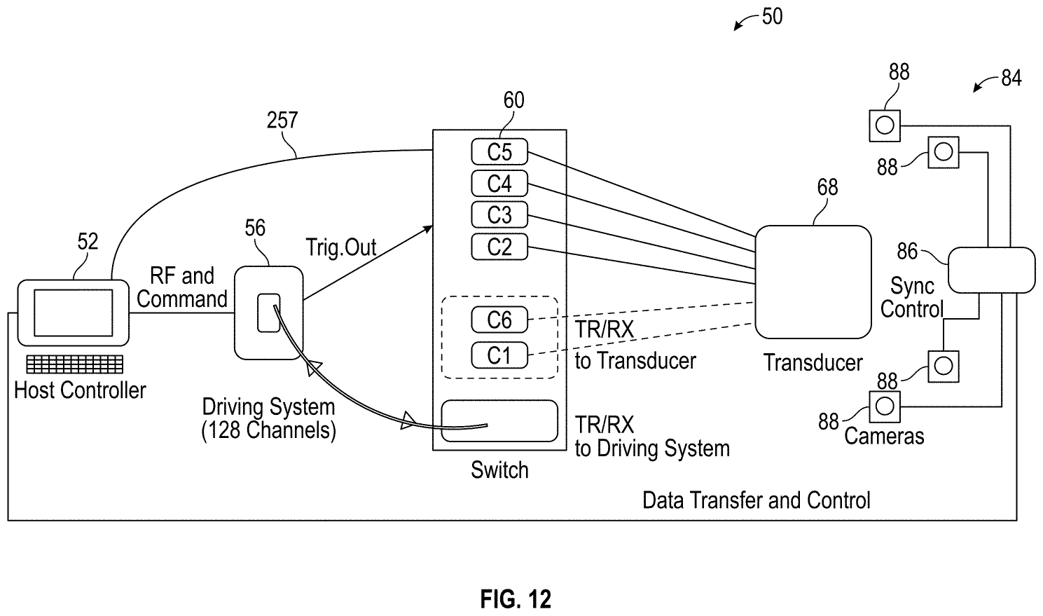

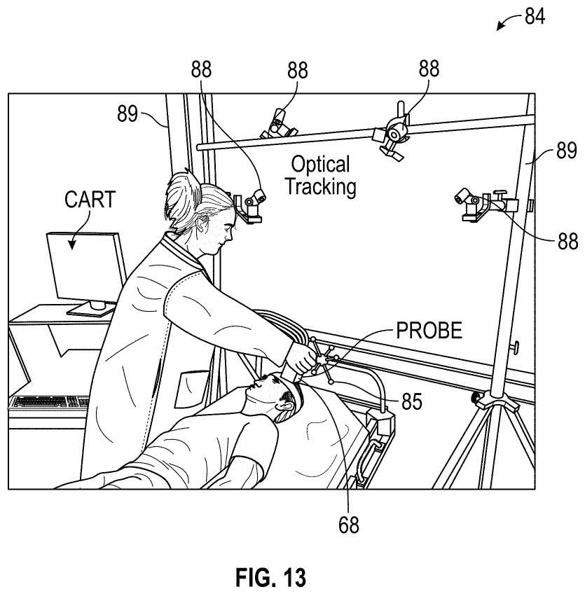

The host controller 52 is a computer that is programmed to control the ultrasound driving system 56 or 90 and, when present, the optical tracking system 84 (FIGS. 12 and 13). The host controller 52 also runs the image reconstruction software to perform image reconstruction based on the digitized received RF signals received from the transducer elements of the ultrasound transducer probe 68 via the ultrasound system switch 60 or 61 and driving system 56 or 90. The image reconstruction algorithms and software in some embodiments may utilize a graphic processing unit (GPU) to rapidly beamform the digitized received RF signals from each transducer element into a three-dimensional (3D) imaging grid. The software may employ a synthetic receive aperture to electronically select which transducer elements contribute to the image (i.e. transducer elements receiving longitudinal waves resulting from transmission at an incident angle to the bone of the subject that produces shear waves or receiving longitudinal waves transmitted at incident angles both above and below the longitudinal critical angle, and/or selecting transducer elements receiving ultrasound waves that have undergone shear mode conversion on the return path to the transducer).

The software 200 (FIG. 11) may selectively apply phase and amplitude correction 222 to digitized receive RF signals 202, depending on the transmit and/or receive incident angle. Filtering 210 may be applied to the digitized receive RF signals received from the transducer elements to remove multiple reflections. The software may apply skull aberration correction 222 based on phase information from the digitized received RF signals; and selective synthetic apertures 214 based on the transmit incident angle of ultrasound (longitudinal and/or shear mode converted transmission) 216 and or the incident angle (longitudinal and/or shear mode converted) of the ultrasound waves returning back through the bone layer following reflection of scattering from features below the bone layer 218. The software may employ contrast and signal enhancement algorithms 206, 228 and 230 to selectively enhance the contrast of weak echoes (reflected longitudinal waves) within the brain. Post processing filters 232 such as edge detection or sharpening filters may be employed. The software may take tracking data 238 to co-register the ultrasound pixels (or voxels) to a global coordinate system, resulting in a larger imaging field of view than what can be produced from one location of the transducer. The tracking data 238 may come from an optical tracking system 84, a magnetic tracking system, a kinetic tracking system or from a software-based tracking 255 which tracks features within the ultrasound image. The images may be displayed as a 2D ultrasound image 242, as a series of 2D slices allowing the operator to scroll through the slices captured for one frame of imaging data, or as a larger montage view, where the software takes the captured images from many positions, along with the positional data from the optical tracking system 84 (FIG. 12) (or magnetic, kinetic or software tracking) to co-register and interpolate a larger montage image 246. In some embodiments, the optical tracking system 84 (or other tracking system) may be employed to enhance the co-registration of images from different locations.

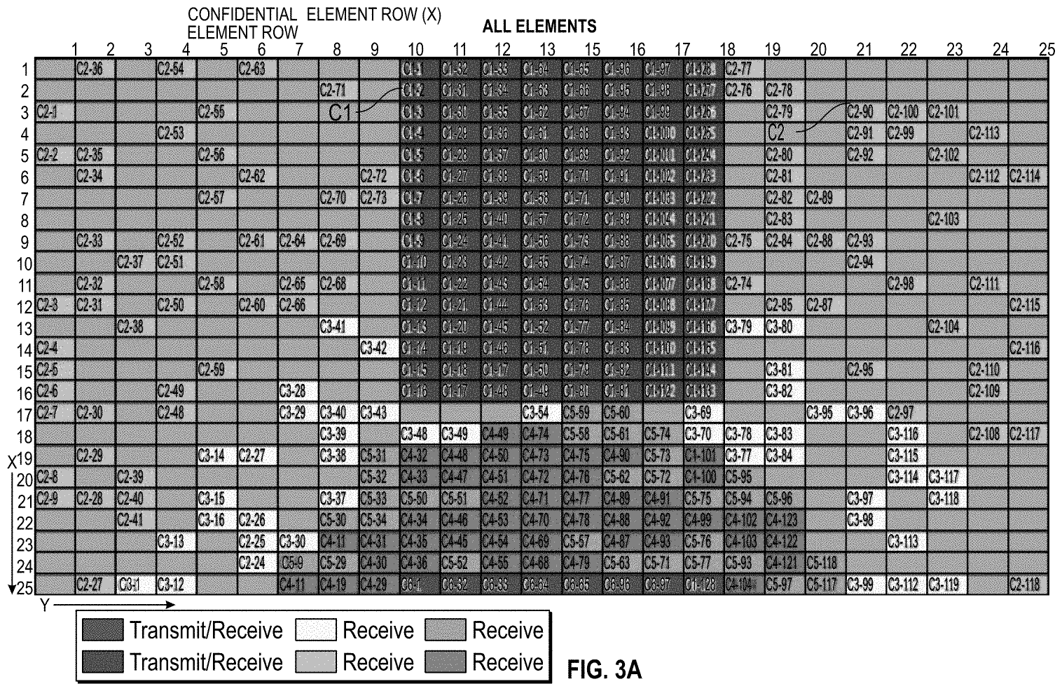

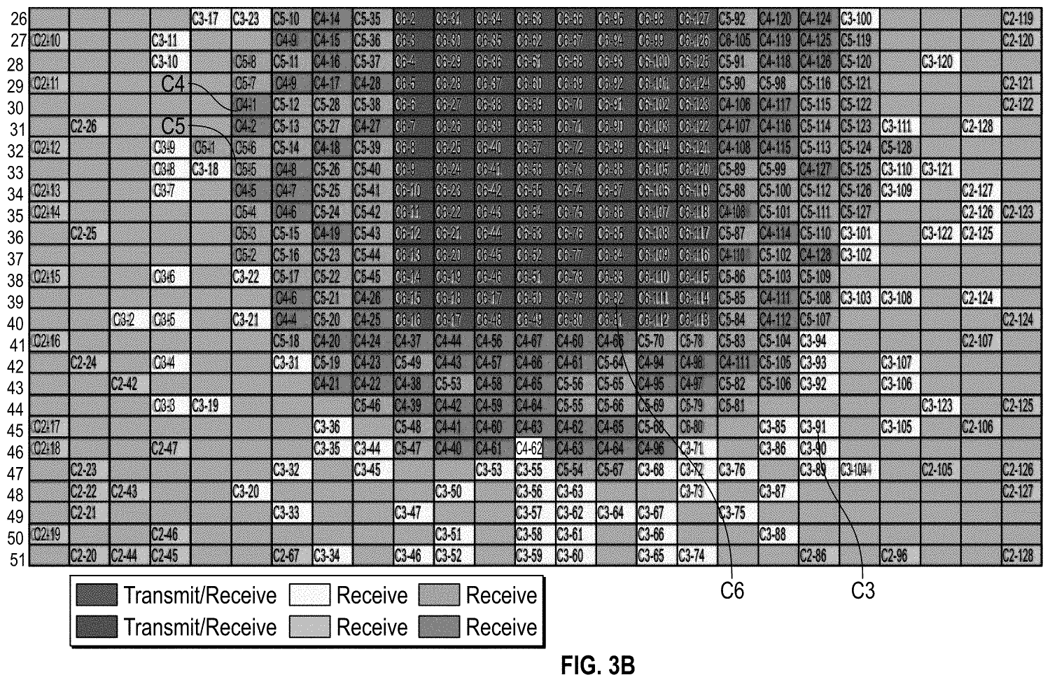

The host controller 52 can manipulate the transmit incident angles 16 for increased field of view (FOV) and optimal received RF signals from both longitudinal waves and converted shear waves. Additionally, or alternatively, the sonographer can achieve greater or different fields of view manually, i.e., by manually repositioning the ultrasound transducer probe 68 on the subject's or patient's head. By positioning the probe 68 in several different positions, whole brain imaging can be achieved. In certain embodiments, received RF signals received from the non-distorted converted shear waves (produced by shear angle transmit/receive pad C1, FIGS. 3A and 3B) and/or received RF signals received from the longitudinal waves are used to correct the phase distortions of the received longitudinal waves. This is different from U.S. Pat. No. 7,175,599 where the phase and amplitude of the transmit beam is corrected in order to improve targeting of delivery of the ultrasound energy to a specific location. In certain embodiments, both transmit/receive pads C1 and C6 are used to transmit ultrasound waves at an incident angle that produces converted shear waves as well as at an incident angle that is near 0° (normal to the plane of the surface of the bone). Thus, embodiments use both longitudinal waves and converted shear waves to produce images rather than using only converted shear waves.

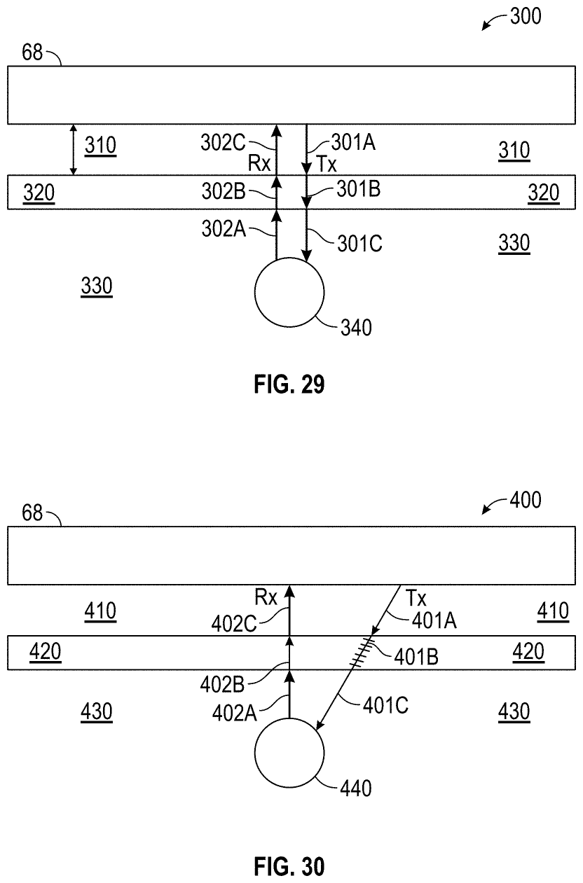

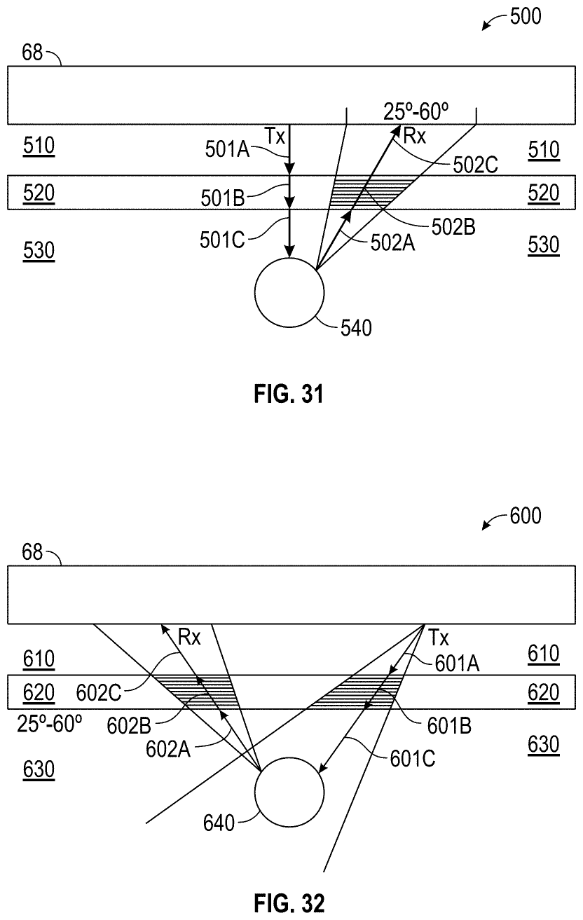

In the dual wave imaging mode, in which longitudinal waves 20 and 36 (FIG. 1) are transmitted towards the skull 10 at various incident angles 16, the reflected longitudinal waves 30 and 46 detected by the transducer elements of the ultrasound transducer probe 68 are any combination of: 1) Zero Wave Conversion—transmitted longitudinal waves that first propagate through the skull, reflect from within the brain, and exit back out through the exterior surface of the skull as longitudinal waves without undergoing any conversion (FIG. 29); (2) Transmit (Tx) Double Wave Conversion—transmitted longitudinal waves that undergo a double conversion as they first propagate through the bone as shear waves, which convert to longitudinal waves upon exiting the interior surface of the bone, which then reflect back at such angle that they propagate back through and exit the exterior surface of the bone as longitudinal waves (FIG. 30); (3) Receive (Rx) Double Wave Conversion—transmitted longitudinal waves that first propagate through the bone as longitudinal waves which then reflect back at such an angle to the interior bone surface that they propagate through the bone as shear waves, which then convert back from shear waves to longitudinal waves upon exiting the exterior surface of the bone (FIG. 31); 4) Quadruple Wave Conversion—transmitted longitudinal waves that first propagate through the bone as shear waves, which then exit the internal surface of the bone and convert to longitudinal waves, which then reflect back from within the brain at such an angle that they propagate back through the bone as shear waves, and then convert back again to longitudinal waves as they exit the exterior surface of the bone (FIG. 32); and 5) Utilizing the transmitted longitudinal waves in any combination of the above scenarios in which any longitudinal waves are immediately reflected back off the bone surface, or trabecular bone, or the interior surface in order to characterize the bone morphology and calculate the phase shift introduced by the bone layer to the propagating longitudinal waves. The longitudinal waves 40 introduced into the brain 11 via shear mode conversion are only effectively/cleanly transmitted to the target region of the object of interest 12 in the brain 11 if transmitted at an incident angle 16 between ˜30 degrees and ˜60 degrees from normal to the plane of the surface of the bone. The transmitted longitudinal waves 20 that have an incident angle below the longitudinal wave critical angle are greatly reflected/attenuated/distorted by the skull 10, but may have the advantage of maintaining greater reflected acoustic power than the longitudinal waves 36 that have an incident angle above the longitudinal wave critical angle, and thus result in shear waves 38. These stronger reflected longitudinal waves 30 are managed via signal processing and image reconstruction software. In some embodiments, the received RF signals collected from the reflected longitudinal waves 30 may be corrected for attenuation and phase shift due to the skull bone 10 for image reconstruction purposes.

FIG. 11 illustrates a process 200 for handling the dual wave ultrasound imaging data generated by the dual wave ultrasound imaging system 50. Particularly, FIG. 11 illustrates the processing for one frame of raw digitized received RF signals 202 from received ultrasound waves detected by the transducer elements of the ultrasound transducer probe 68. The process 200 includes a preprocessing module 204, a data selection module 214, an image reconstruction module 220, a post processing module 226, a visualization module 240, and optionally a four-dimensional (4D) visualization module 254 based on the receipt of multiple frames resulting from the process 200. In the preprocessing module 204, the process 200 receives one frame of digitized received RF signals received by the transducer 202. The preprocessing module 204 enhances the depth of the image at step 206, filters the digitized received RF signals to reduce skull reflections at step 210, determines the skull characteristics at step 208, and estimates phase shift at step 212 based on the determination of skull characteristics at step 208.

From the preprocessing module 204, the process 200 proceeds to the data selection module 214. At step 216 in the data selection module 214, the process selects the transmit event based on the identified transmit pad and the steering angle or transmit incident angle. At step 218, the process generates and applies a synthetic receive aperture based on the identified receive pad, and the receive incident angle upon the bone layer and the transducer.

From the data selection module 214, the process 200 proceeds to the image reconstruction module 220. At step 222 of the image reconstruction module 220, the process performs aberration correction and at step 224, the process performs beamforming to the three-dimensional ultrasound grid.

From the image reconstruction module 220, the process 200 proceeds to the post processing module 226. At step 228 of the post processing module 226, the process enhances the contrast of the image. At step 230 the process employs depth enhancement to the ultrasound image. At step 232, the process employs filters to further enhance the ultrasound image. At step 255 the process tracks features within frames of ultrasound imaging data to produce tracking data 238. At step 234, the process receives tracking data 238 from the tracking system (optical tracking 84, or magnetic, kinetic or software tracking 255). With the tracking data 238, the process at step 234 co-register's the ultrasound pixels (or voxels) to a global coordinate system. At step 236, a 3D montage image of the whole brain is created from the co-registered ultrasound pixels (or voxels).

From post processing module 226, the process proceeds to the visualization module 240. At the visualization module 240, the following processes may be employed within the module 240. At step 242, the process creates a 2D ultrasound image from one slice of the imaging volume for one field of view (FOV). At step 244, the process creates 3D orthogonal slices of the ultrasound imaging volume for one field of view of the transducer (i.e. one frame). At step 248, the process creates a 2D ultrasound image of the whole brain montage (from process 236). At step 250, the process creates a 3D tomographic image of the ultrasound image from one FOV. At step 252, the process creates a 3D tomographic image from the whole brain montage from the interpolated and co-registered ultrasound voxels of steps 234 and 236. At step 246, the process creates whole brain 3D orthogonal slices of ultrasound imaging data from the output of process 236. At step 252, the process creates a whole brain 3D tomographic image 252 from the output of process 236.

### Ultrasound Transducer Probe

With reference to FIGS. 3A and 3B, the ultrasound transducer probe 68 may be a 25×51 array of 1275 transducer element positions for the piezo electric crystals. In certain embodiments, rather than transmitting and receiving ultrasound signals via a single array with transducer elements (such as piezo electric crystals) serving as both transmit and receive channels, ultrasound waves are transmitted using separate transmit and receive arrays or pads. In FIGS. 3A and 3B, transmit/receive pads C1 and C6 with transducer elements produce transmitted longitudinal ultrasound waves. Separate receive arrays or pads C2, C3, C4, and C5 are employed to receive reflected longitudinal ultrasound signals. Receive pads C2, C3, C4, and C5 have been configured to improve signal to noise ratios (SNR) and overall resolution. Of the 1275 transducer element positions shown in FIGS. 3A and 3B, 507 elements are left unconnected resulting in 768 active piezo electric crystal elements. The active piezo electric crystal elements are arranged into transmit/receive pads (elements C1-1 through C1-128 and elements C6-1 through C6-128), receive pads (elements C2-1 through C2-128, elements C3-1 through C3-128, elements C4-1 through C4-128, and elements C5-1 through C5-128). The hand-held ultrasound transducer probe 68 transmits or receives longitudinal waves at various incident angles so that the longitudinal ultrasound waves 20 or 36 (FIGS. 1A and 1B) propagate through the skull 10 either as shear waves 38 or as longitudinal waves 22. As shown in FIGS. 3A and 3B, the ultrasound transducer probe 68 is sparsely populated with receive elements 1-128 in receive pads (C2, C3, C4, and C5) arrayed around the transmit/receive pads (C1 and C6) to maximize detection of longitudinal waves 30 and 46 that have reflected within the brain and propagated back through the skull 10 either as shear waves 44 or as longitudinal waves 28 (FIGS. 1A and 1B).

In certain embodiments of the ultrasound transducer probe 68, the ultrasound transmit transducer elements (piezo electric crystals) are configured into two sets of pads C1 and C6. In FIGS. 3A and 3B, one transmit/receive pad C6 is centrally positioned and is configured to transmit longitudinal waves at an incident angle of near 0° (normal) 16 into the skull 10, and the other transmit/receive pad C1 is offset from the center of the ultrasound transducer probe 68 and is configured to transmit at an optimal incident angle 16, namely within Snell's critical angle window for shear mode conversion (30 degrees-60 degrees from normal to the plane of the surface of the bone), via configurable beam steering. The longitudinal transmit/receive pad C6 is generally positioned at the center of the transducer array, to take advantage of the surrounding receive elements C2, C3, C4, and C5 when transmitting at an incident angle of near 0° (normal), while the “shear angle transmit” pad C1 is generally positioned off-center so that the longitudinal transmit/receive pad C6 may act as a receive pad for the reflected longitudinal waves. In certain embodiments, either or both of the transmit/receive pads C1 and C6 are capable of transmitting at an incident angle 16 of near 0° (normal) 10, and at an incident angle 16 that produces shear waves within the skull 10 (between 25 degrees and 60 degrees to normal). In other embodiments, additional transmit/receive pads may be included to increase the imaging volume and field of view.

In certain embodiments, such as the ultrasound transducer probe 68 shown in FIGS. 3A and 3B, the receive pads C2, C3, C4, and C5 are arranged around the transmit/receive pads C1 and C6 and are sparsely populated pads (e.g., with a randomized distribution of elements to maximize the variations in distances between elements, in order to reduce artifacts). Among other things, the sparsely populated receive pads C2, C3, C4, and C5 allow for reception from distorted, yet stronger, reflections from ultrasound waves originating from the longitudinal wave transmit/receive pad C6 that have passed back through the skull layer as longitudinal waves, and as shear waves. The location of receive pads C3, C4, C5 and transmit/receive pad C6 is configured to increase the density of receive elements along the direction of the incident angle (or steering angle) to produce a shear wave converted ultrasound beam originating from transmit/receive pad C1 in order to improve the detection of the reflections of those waves. Specific patterns of receive pads are described below although other optimal or acceptable patterns can be determined for a particular application without undue experimentation using techniques described herein.

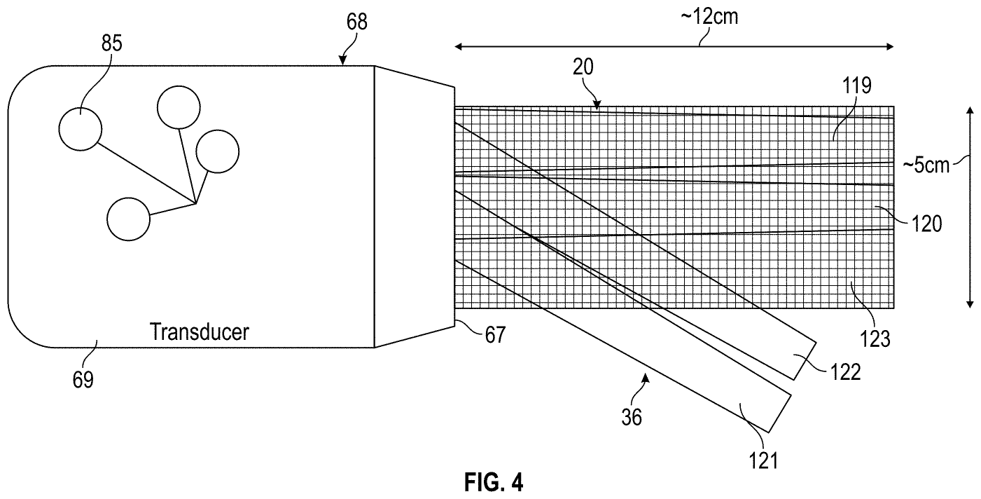

The hand-held probe 68 includes a housing 69 (FIG. 4). The probe housing 69 contains the 768 (6 pads×128 elements) active ultrasound transducer elements that are used to transmit and receive ultrasound signals. The transducer element bandwidth is greater than 82% per transducer element. The resolution ˜1.2 microseconds. The central frequency is 970 kHz. The element pitch is ˜0.95×1.9 mm with a kerf less than 100 microns. These specifications could vary. With reference to FIG. 4, the transducer 68 is shown with the following transmit beams: normal incidence transmit beam 119 from pad C1, normal incidence transmit beam 120 from pad C6, shear angle transmit beam 122 from pad C1, and shear angle transmit beam 121 from pad C6. The angle of transmit is along the x axis, as shown in FIGS. 3A and 3B. A 2D image reconstruction grid 123 is also shown. Other transmit schemes may be employed.

As previously discussed, FIGS. 3A and 3B show the design of the transducer elements of the ultrasound transducer probe 68 in accordance with the present invention. The ultrasound transducer probe 68 includes two 128-element transmit/receive pads C1 and C6 that can be used for longitudinal and shear mode propagation through the skull bone and also includes four 128-element sparse receive pads C2, C3, C4, and C5 with density increasing along the direction of the transmit incident angle for shear mode conversion within the bone layer.

The ultrasound transducer probe 68 is waterproofed up to the back connectors, with a maximum leakage current of 50 uA from the front face 67. The transducer housing is constructed from polyetheretherketone (PEEK) for durability, and the front face 67 is covered with a thin layer of silicone. A gel pad assembly (e.g., a 2 cm thick gel pad, not shown) may be used for acoustic coupling between the probe transducers and the subject's head.

### Driving System

In one embodiment shown in FIG. 5, a commercially available ultrasound driving system (also known as ultrasound transceiver) 56 with at least 128 channels produces transmit RF signals which are passed through ultrasound system switch 60 to drive the transmit transducer elements of the ultrasound transducer probe 68 to produce the longitudinal waves 20 and 36. The driving system 56 captures the received RF signal transmitted through ultrasound system switch 60 and created by the reflected longitudinal waves received by the transducer elements of probe 68. The driving system 56 is connected to the ultrasound system switch 60 by two-way connection 58 and switch input connector 59.

In another embodiment shown in FIG. 7, a commercially available ultrasound driving system 90 with at least 256 channels produces transmit RF signals to drive the transmit transducer elements of the ultrasound transducer probe 68 to produce the longitudinal waves 20 and 36. The driving system 90 captures the received RF signal transmitted through ultrasound system switch 61 and created by the reflected longitudinal waves received by the transducer elements of probe 68. The driving system 90 has a connector 91 and connector 92. The connector 91 is connected either directly, or through an adapter cable 96, to the transmit/receive pad C1 of the ultrasound transducer probe 68, and the receive connector 92 is connected to the input connector 93 of the receive switch 61 via the cable 94.

The ultrasound driving system 56 or 90 receives the received RF signals from the transducer 68 via the ultrasound system switch 60 or 61, and then performs analog and signal digital processing to produce digitized RF signals to be used by the Host controller 52 to reconstruct the ultrasound images using process 200.

A shear angle transmit pad, such as C1 (FIGS. 3A and 3B), may be fabricated by changing the orientation of the pad within the transducer, rather than only via electronic steering.

### Ultrasound System Switch

The ultrasound system switch is an electronic component devised for establishing connection between the transducer elements and the electronic channels of the ultrasound driving system in order to allow the transfer of RF signals. A large number of transducer array elements may be employed for transmitting and receiving ultrasound waves, up to and including all transducer array elements (sometimes referred to herein as a “fully populated” array or pad). The number of channels supported by a third-party ultrasound driving system, however, is less than the number of transducer array elements used by the ultrasound transducer probe 68 for receiving ultrasound waves. Therefore, the transducer array elements used for receiving longitudinal ultrasound waves are configured to include multiple (e.g., four, five, or six) pads of receive elements, with a switching system used to selectively couple the receive pads to any particular third-party ultrasound driving system 56 or 90 in order to capture a larger number of receive transducer elements.

The ultrasound system switch 60 (FIG. 5) or 61 (FIG. 7) allows the probe to be integrated with the third-party driving systems 56 and 90 that have fewer channels than the number of transducer elements of the ultrasound transducer probe 68. The ultrasound system switch 60 provides rapid switching between channels to occur immediately following the transmit event, to allow transmit and receive on separate transducer elements of the ultrasound transducer probe 68.

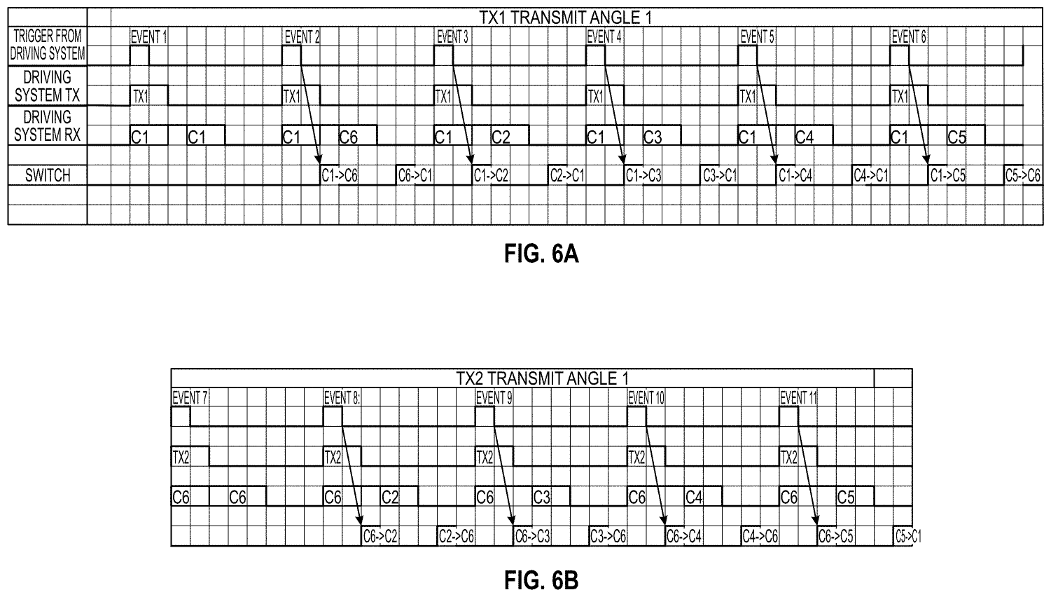

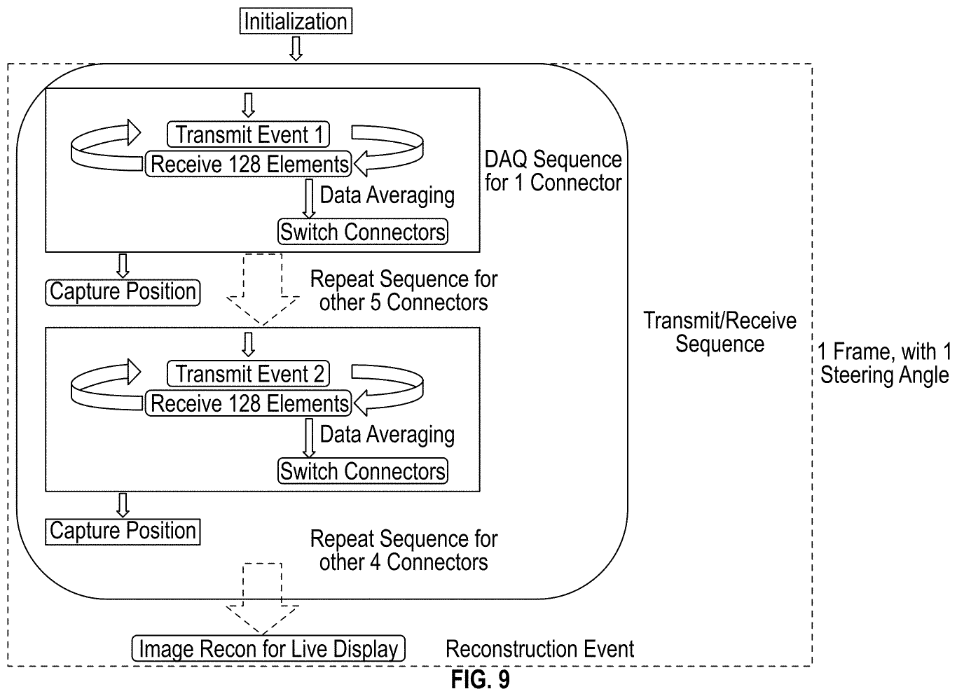

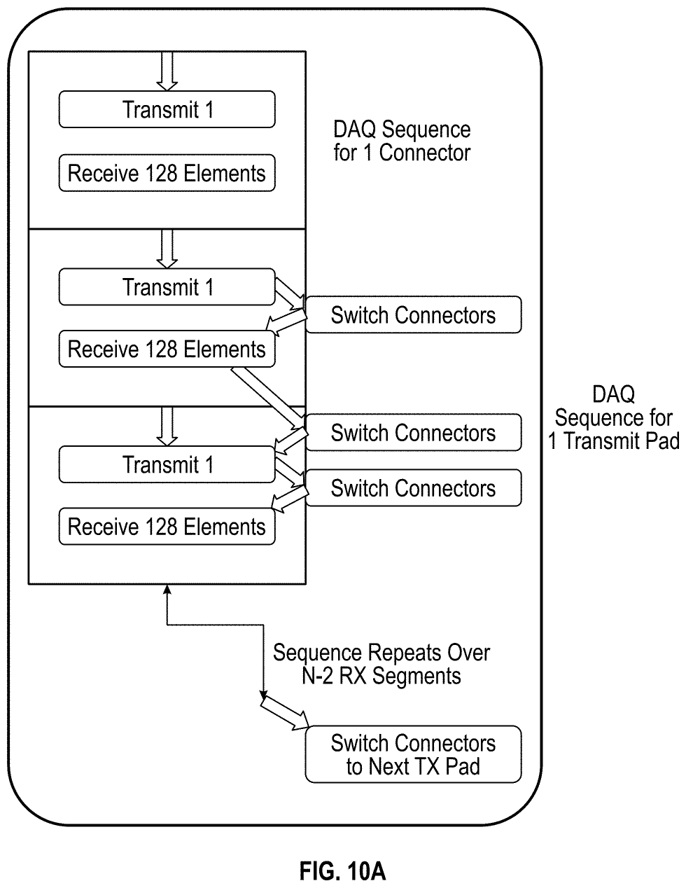

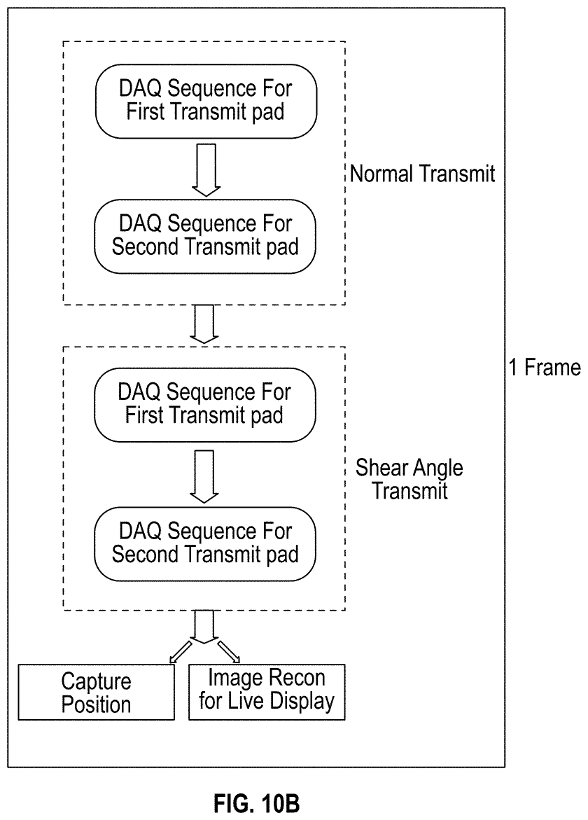

In FIG. 5, the six 128 channel transducer pads C1, C6, C2, C3, C4, and C5, of the ultrasound transducer probe 68 are connected to the six distinct transmit/receive interface connectors T1, T6, R2, R3, R4, and R5 of the ultrasound system switch 60 via probe connectors N1, N2, N3, N4, N5, and N6. The one common 128 channel transmit/receive interface connector 59 of the switch 60 is connected to one of the 128 channel ultrasound driving system connectors of the driving system 56. The ultrasound system switch 60 is powered from a 120 VAC line 13 from which +5 VDC 14 and +3.3 VDC 15 are produced. A communications interface such as a USB, Ethernet connection or similar 257 between the ultrasound system switch and the host controller allows for command and status signals to be communicated between the ultrasound system switch and the host controller. Each transmit event occurs with the appropriate transducer transmit/receive pad C1 or C6 of the ultrasound transducer probe 68 connected electronically through the ultrasound system switch 60. With reference to FIGS. 6A and 6B, the first transmit event is received on the same elements that were used to transmit the ultrasound waves. For the following transmit events, approximately 10 microseconds after the transmit event occurs, the ultrasound system switch 60 disconnects the transducer transmit/receive pad C1 (or C6 in the case of FIG. 6B) and connects one of the five other transducer pads to receive the longitudinal ultrasound wave echo captured by that pad. Each transmit event is repeated with the ultrasound system switch selecting the appropriate receive pad based on a programmable predetermined sequence to allow the received RF signals of the reflected longitudinal wave signal received from the 640 or 768 transducer elements of the ultrasound transducer probe 68 of FIGS. 3A and 3B. One example of the data acquisition sequence can be seen in FIGS. 6A and 6B. This sequence may be changed. FIG. 9 shows the data acquisition (imaging) sequence for one frame of imaging data, where averaging is employed to improve the SNR for each pair of transmit pad/receive pad employed, and where one transmit mode (incident angle) is chosen per frame. FIGS. 10A-10B show an imaging sequence in another embodiment, where both normal transmit (incident angle near 0°) and shear angle transmit events contribute to one frame of imaging data. More or fewer transmit incident angles could be employed in another embodiment of the dual wave ultrasound imaging system 50. More or fewer channels may be accommodated by modification of the ultrasound system switch design.

A receive-only ultrasound system switch 61 (FIG. 7) is used to allow switching between receive elements of pads C2, C3, C4, and C5, on the probe 68, for an ultrasound driving system 90 with only 256 channels. Each of the transmit/receive pads C1 and C6 of the ultrasound transducer probe 68 is swapped between the first 128-channel interface 91 of the ultrasound driving system 90. Four of the receive connectors R2, R3, R4, and R5 connect to the ultrasound driving system 90 through the receive only ultrasound system switch 61. In an embodiment, each transmit event was repeated four times to allow the received RF signals generated by the transducer elements on 640 transducer elements to be captured. In certain embodiments, manual switching between the two transmit/receive pads C1 and C6 can be implemented, although a 256:128 ultrasound system switch or other switching arrangement may be used in various alternative embodiments to switch between the two transmit/receive pads C1 and C6 of the ultrasound transducer probe 68.

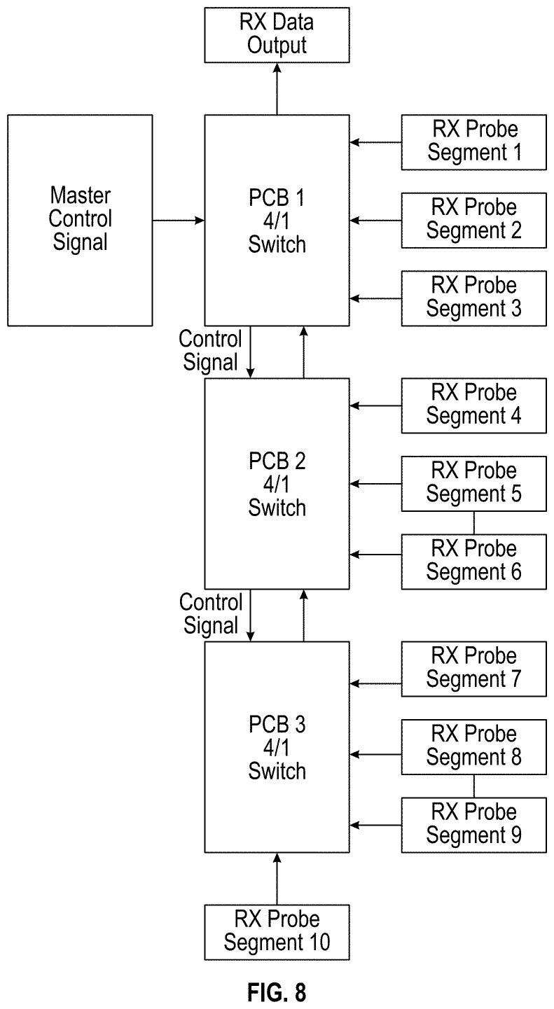

In certain embodiments, the ultrasound system switch 61 includes Individual 512:128 channel multiplexor printed circuit boards (PCBs) that can connect three 128 probe element pads (e.g., using 260 position ITT Cannon DL Series ZIF connectors) to one 128 channel connector. In certain other embodiments, these PCBs can be daisy chained to create a 128*(3*N+1):128 channel multiplexor, where N is the number of PCBs connected, in order to connect 3*N+1 probe pads consisting of 128 elements to the one 128 channel transmit/receive connector of the ultrasound driving system. FIG. 8 is a schematic diagram showing the switching system architecture with three 512:128 PCBs to create a 1280:10 switch. FIG. 7 shows the block diagram of the 512:128 receive only switch 61, in accordance with certain embodiments. In certain embodiments (illustrated in FIG. 5), the circuitry of the ultrasound system switch 60 is fabricated to include a commercially-available 16 channel high voltage analog switch (16 HV2733 ICs per PCB for a total of 128 SPDT switches) with a PIC32MZ2048ECG embedded microcontroller to control the switching of the HV2733 ICs. The PIC32 embedded microcontroller controls which HV2733 multiplexor is active. Switching time can be driven by an external trigger or provided by the PIC32 embedded microcontroller to synchronize the switches with the transmit/receive events of the driving system. The timing diagram for one embodiment of the ultrasound system switch 60 is shown in FIGS. 6A-6B.

The ultrasound system switch 60 or 61 allows the ultrasound transducer probe 68, which contains a plurality of transducer elements, to establish electrical connection with the driving system 56 or 90 that has fewer channels than the ultrasound transducer probe 68. The ultrasound system switch 60 is designed to include circuitry to limit the ways in which voltage is applied to the ultrasound transducer probe 68, preventing the ultrasound system switch 60 from transferring energy to the probe 68 in an unexpected manner. The ultrasound system switch 60 includes fuses to limit the maximum voltage applied to the ultrasound transducer probe 68. Certain channels of the ultrasound system switch 60 are monitored to ensure the transmit RF signals have completed within the allotted time before switching to the receive channels. In the one embodiment, the ultrasound system switch 60 will not allow a transmit RF signal that persists beyond the specified duration to transmit to the ultrasound transducer probe 68, in order to mitigate the risk of uncontrolled acoustic output from the ultrasound transducer probe 68. The circuitry is designed to rapidly switch to another segment of the ultrasound transducer probe 68 immediately after transmit, allowing the received RF signal from any element to be captured regardless of the transmit RF signal pattern used. This allows the same electrical channel from driving system 56 to be used for transmit and receive on separate transducer elements for a single transmit event. Inclusion of the ultrasound system switch allows the ultrasound transducer probe 68 of the dual wave ultrasound imaging system 50 to be used with a variety of third-party driving systems 56 and 90 without necessarily needing special customization of the driving system.

The user may implement any desired image processing algorithm by accessing the digitized received RF signals captured by the driving system and produced by the transducer elements which are mapped by the ultrasound system switch 60 or 61 to a composite data set of digitized received RF signals which includes all connected transducer elements, regardless of the number of electrical channels available in the driving system 56 or 90. In the disclosed embodiment, the switching sequence is TxC1/RxC1 (no switching), TxC1/RxC6, TxC1/RxC2, TxC1/RxC3, TxC1/RxC4, TxC1/RxC5, TxC6/RxC6 (no switching), TxC6/RxC2, TxC6/RxC3, TxC6/RxC4, TxC6/RxC5, where Tx denotes the transmit pad of the ultrasound transducer probe 68 used, and Rx denotes the receive pad of the ultrasound transducer probe 68. The switching sequence can be changed via a firmware update to the ultrasound system switch, and other sequences may be used. A switch signal can be detected on the RF signal line 58 (FIG. 2) prior to the return of the first reflected longitudinal wave echo from the patient. In some embodiments this signal is used to verify the switching sequence by the image reconstruction software.

The Tx/Rx ultrasound system switch 60 (FIG. 5) consists of 128 six to one (6:1) or more or fewer analog switches (Maxim Integrated MAX14866) that are simultaneously activated to allow one of up to six ultrasound transducer connectors (each containing 128 ultrasound elements) to be connected to ultrasound system switch output connector. Power for the switching circuitry consists of two voltage rails of +5, and +3.3 Volts, each drawing no more than 1 Amp. Logic and timing signals of the ultrasound system switch are controlled by a PIC32MZ2048EFH100 microcontroller. The ultrasound system switch consists of switching elements capable of switching high voltage transmit signal (e.g. +/−100V) between six contact receptacles from one contact receptacle. Switching speed must be fast enough to allow the transmit pulse to be sent through to one 128 channel transducer connector and receive on a different connector. The microcontroller uses internal timers to ensure that switching occurs only after the transmit pulse is expected to complete. The 128 channel 6 to 1 analog ultrasound system switch unit consists of a custom made printed circuited board (PCB), indicator LEDs, and multiple AC/DC switching power supplies housed in an aluminum chassis. The ultrasound system switch is designed to accept up to 128 high voltage analog signals on each of its 6 input connectors, as well as having two TTL trigger signal inputs, a mini USB connector, and an AC power inlet. The maximum allowable voltages on these inputs are as followed:

ANALOG INPUTS:

±100 Volts Signal Input Max.

TTL INPUTS:

0 to +5 Volts Max input.

50 Milliamps Current Max per input.

MINI USB:

Standard Mini USB Connection to Host Device. USB 2.0 Hi Speed Compliant.

AC POWER:

88 o 125 VAC Max input.

IEC 320-C14 Compliant Power Entry Inlet

Alternative ultrasound system switches may include more or fewer receive channels, more or fewer PCBs or switch arrangements (e.g., a single 512:128 switching matrix), more or fewer channels per interface, different microcontroller, similar switch components, etc. In certain embodiments, where the number of channels of the driving system is equal to the number of active transducer elements in the probe, the ultrasound switching system would be utilized as a safety measure to prevent uncontrolled acoustic exposure, by limiting the transmit voltage applied to the transducer through fuses and by limiting the duration of the transmit RF pulse through channel monitoring, where the switch would disconnect from the transducer in the event of a transmit RF pulse that is longer than the expected duration. The switching logic of the type described herein may be included in the ultrasound probe rather than using one or more separate switches. For example, the ultrasound probe may have one receive output interface that is switched internally between multiple receive pads, e.g., under the control of the host controller.

### Position Tracking

With reference to FIGS. 12 and 13, in certain embodiments, the position of the ultrasound probe on the patient's head is monitored (e.g., tracked optically, acoustically, magnetically, etc.), where the positional information is used to co-register ultrasound pixels (or voxels), or otherwise correlate with imaging data from the ultrasound probe 68 in order to enhance imaging and image processing capabilities of the dual wave ultrasound imaging system 50 (e.g. to allow orthogonal and tomographical views of the brain, similar to current CT and MM post-processing image viewing).

In certain embodiments that utilize an optical tracking system 84, the optical tracking system is a commercially available optical tracking system that is used by the host controller to track the position of the probe in order to correlate the position of the ultrasound transducer probe 68 with digitized receive RF signals received via the ultrasound driving system 56 or 90, (e.g., to create montage images 236 from multiple frames or to create 3D images from multiple 2D frames). In other embodiments, the tracking system 84 is a commercially available magnetic tracking system, or additionally kinetic tracking combined with software tracking.

In order to coordinate ultrasound imaging, the hand-held ultrasound transducer probe 68 is tracked via the commercially available passive optical tracking system 84. The optical tracking system 84 (FIG. 12, FIG. 13) includes four infrared cameras 88 attached to a camera frame 89 adjacent the patient. More or fewer cameras may be used. A tracking body 85 (FIG. 4) is attached to the housing 69 of the ultrasound transducer probe 68. The transform between the ultrasound imaging matrix and the tracking body 85 is determined by imaging needles scanned through a variety of locations with a 3-axis positioner scanning arm. Both the needle and the ultrasound transducer probe 68 are optically tracked in order to determine the transform between the ultrasound pixels (or voxels) and the tracking body 85 fixed to the housing 69 of the ultrasound transducer probe 68. The outputs of the cameras 88 are fed to a sync control unit 86 that controls the camera synchronization and data stream between the cameras and the host controller 52. The camera data is fed to the host controller 52 to calculate the position and orientation of the ultrasound transducer probe 68 with respect to the skull 10 of patient. The position information is used in connection with imaging by the host computer 52.

### Testing

The ultrasound transducer probe 68 was tested in silico (computer simulation) for beamforming performance over a series of steering angles (0-45°) and a series of frequencies (455, 700, 800 and 900 kHz and 1 MHz). The geometries capable of producing minimal side lobes were chosen as the candidate transmit geometries to be further tested with the receive geometries in order to determine and optimize transcranial imaging performance. Simulations were carried out using the k-space corrected pseudo-spectral time domain (PSTD) from the k-wave simulation toolbox [29-32]. Sensors were placed in a grid formation in the transmit plane and reconstructed into individual receive elements in order to test receive geometries. By recording the received RF signals for each transducer element, a single simulation can be used to assess the performance of many different receive geometries for the same transmit parameters and imaging medium. The received RF signal for each transducer element was used to reconstruct single wave, shear angle, and dual wave imaging mode reflection mode images. Using this approach, over 800 test cases were generated to optimize transmit and receive geometries along with the optimal frequency, minimum acceptable bandwidth, element sensitivity, element size, and layout.

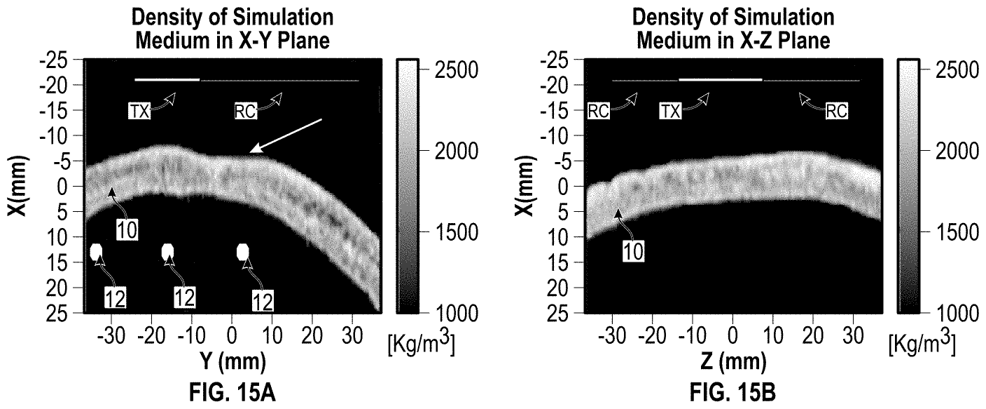

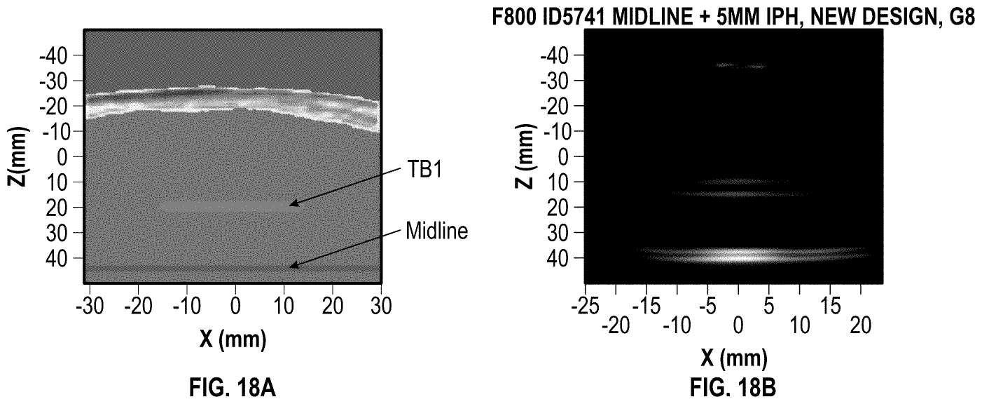

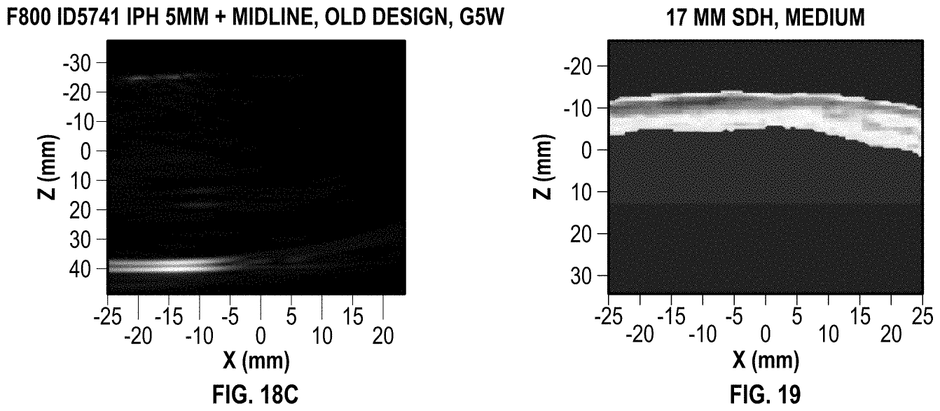

The performance of candidate transducer element array designs were tested using simulations of imaging through skull bone (FIG. 18A), where the simulation medium was taken from CT scans of skull caps. The density of the skull bone is determined from a simple relation between the CT pixel intensity in bone (in Hounsfield units, HU), air and water, given by the relation: HU=1000*(μ−μ_water)/(μ-μ_air). The acoustic properties of bone are then calculated based on the density of bone for a given pixel and the frequency of the transducer, extrapolated from the results reported by [15], where the frequency dependence of the acoustic properties were extracted for several skull caps [24]. The shear wave speed was determined to be lower by a fraction of 1400/2700, and the shear wave attenuation was higher by a fraction of 90/85 [33 34]. FIGS. 15A and 15B show a map of the densities, taken from the skull cap measurements, used in one of the simulation batches for a 700 kHz grid test using the measured densities of a skull cap as the skull phantom, where FIG. 15A shows the x-y plane at Nz/2 and FIG. 15B shows the x-z plane at Ny/2. The transmit element zone and the receive element zone are identified as Tx and Rc in FIGS. 15A and 15B. FIG. 15A further shows objects 12 (bone fragments) to simulate objects such as shrapnel producing traumatic brain injury. The transmit element zone is identified by Tx and the receive element zone is identified by Rc in FIGS. 15A and 15B.

In silico prototype performance of ultrasound transducer probe 68 were tested on several heterogeneous skull cap fragments, where the simulation medium was matched to the modelled acoustic parameters of the imaged skull cap.

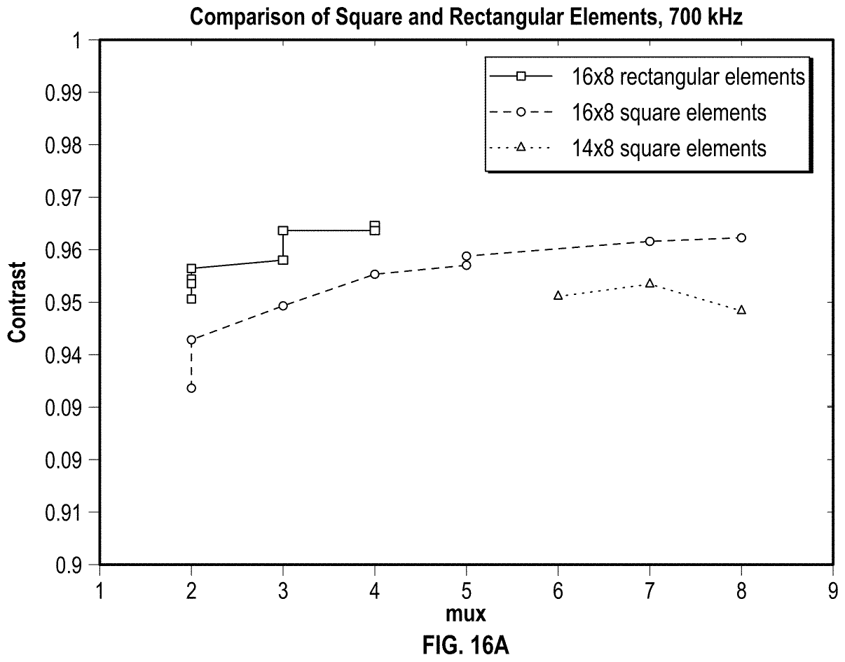

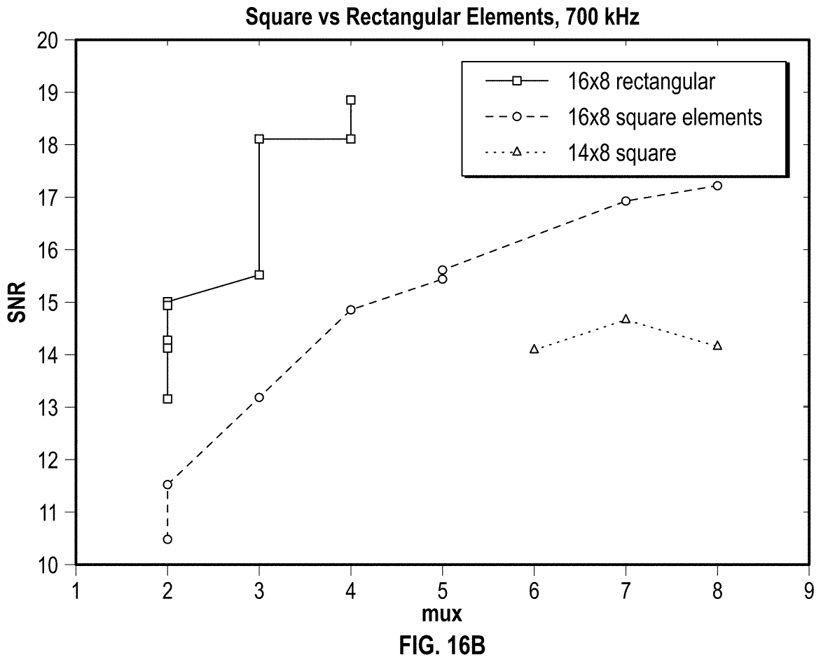

Using these simulated cases, the trade-off between image quality and number of channels was investigated. The motivation for moving to a sparse array of receiver elements is to reduce the cost of the imaging system, and to improve imaging speed, due to a fewer number of electronic channels. FIGS. 16A and 16B show an example of the in silico prototype performance testing carried out for two candidate element geometries for the ultrasound transducer probe 68. In the example shown, the performance of square and rectangular element arrays were tested with a transmission frequency of 700 kHz. The receiver element density is expressed as the multiplexing that would be required for a 128 channel ultrasound driving system. The contrast (FIG. 16A) and signal-to-noise ratio (SNR) (FIG. 16B) were evaluated for several candidate geometries to establish the performance enhancement as a function of array design. In this example, candidate transmit geometries of 16×8 and 14×8 square elements at half wavelength pitch and 16×8 rectangular elements were evaluated with a central transmit frequency of 700 kHz.

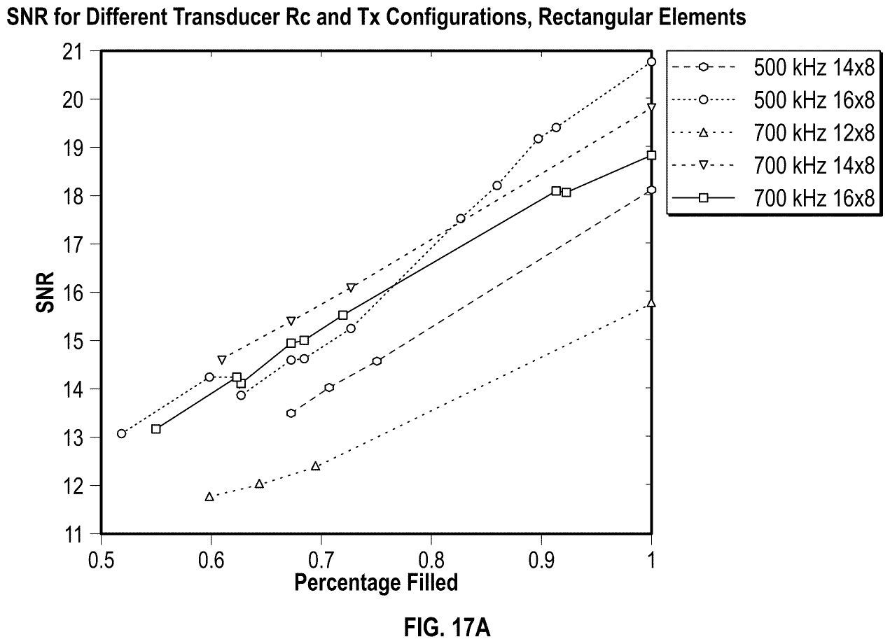

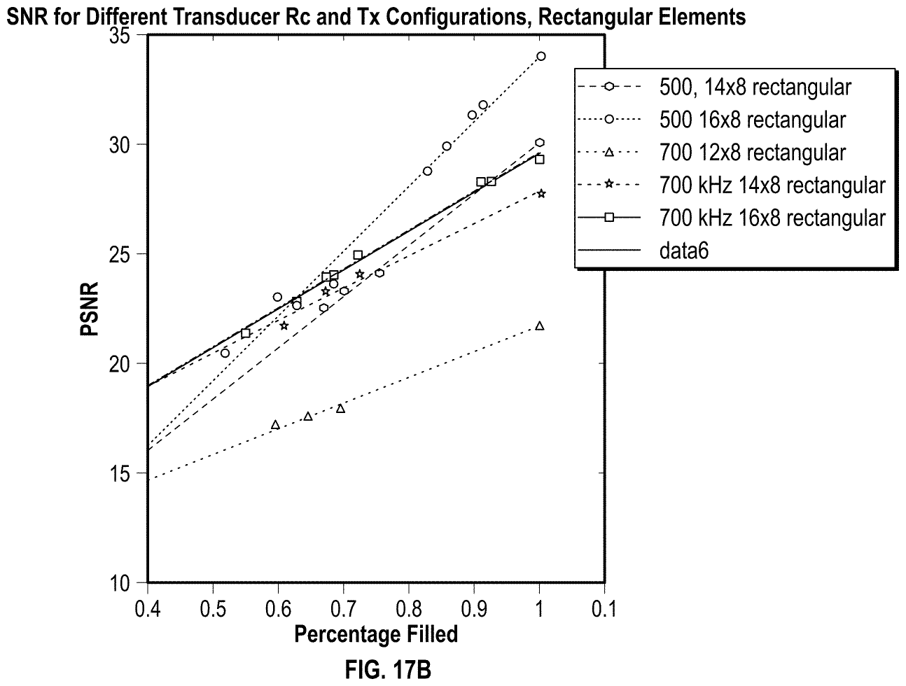

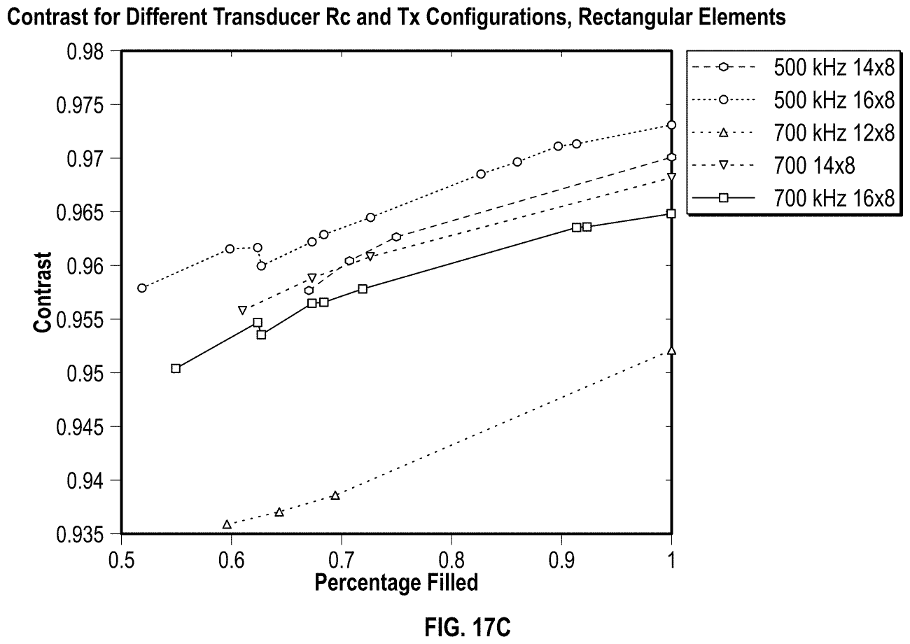

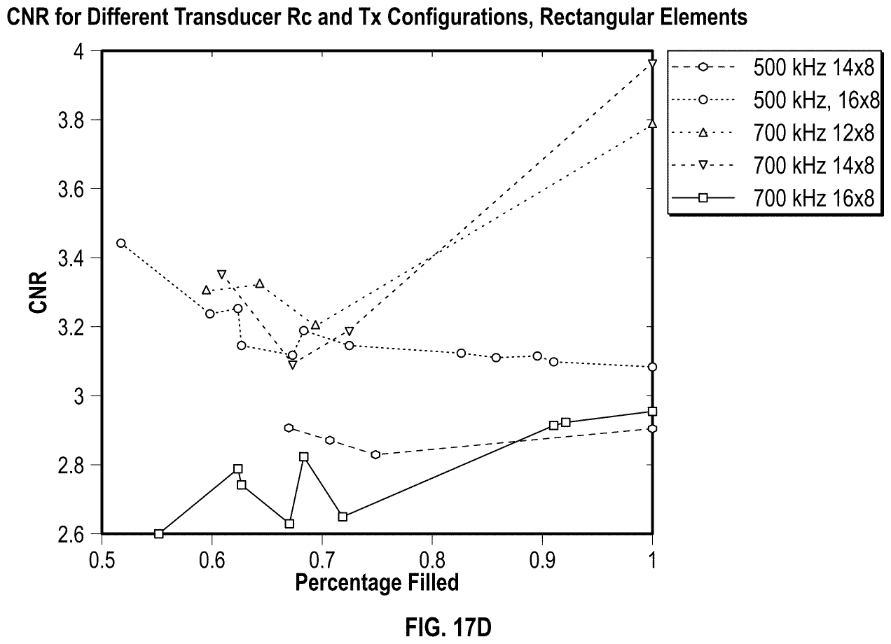

A wider range of frequencies for several candidate transducer element geometries that were tested is shown in FIGS. 17A-17D. The performance SNR (FIG. 17A), Peak SNR (FIG. 17B), contrast (FIG. 17C), and contrast to noise ratio (CNR) (FIG. 17D) were evaluated as a function of percentage of array population for several array geometries at 500 and 700 KHz. The test medium included shrapnel below the skull cap and was kept constant between test geometries. Image quality parameters were assessed for the pixels comprising this bone fragment and compared to a region of interest with no features.

A series of skull caps with simulated traumatic brain injury (TBI) such as epidural hematoma, subdural hematoma, interparenchymal hematoma (ranging from 0.3 cc-20 cc), bone fragments, steel shrapnel, and midline shift were tested for each candidate geometry in order to finalize and validate transducer design. Hematoma sizes and midline shift were chosen based on the recommendation for surgical intervention as described by the American Association of Neurological Surgeons [35-36]. The acoustic properties of brain tissue, and blood for simulated TBI, were taken from Duck et al [39] and Goss et al [40] and are shown in Table 1:

TABLE 1
ACOUSTIC PARAMETERS

| | | Attenuation | | | |
|---|---|---|---|---|---|
| | | (dB/(MHz\hat{ }2 cm)) | | | |
| | Sound Speed | 500 | 700 | 800 | 1 |
| Tissue | (m/s) | kHz | kHz | kHz | MHz |
| Blood | 1584 | 0.1840 | N/A | .072 | 0.69 |
| Brain | 1540 | 1.20 | 0.56 | 0.793 | 0.715 |
| Cerebrospinal | 1507 | 0.184 | N/A | .072 | .069 |
| fluid (CSF) | | | | | |

Signal to noise ratio (SNR), contrast to noise ratio (CNR), and contrast were the metric of evaluation. FIGS. 18A-18C, 19, and 20A-20B show imaging capabilities for the geometry of the ultrasound transducer probe 68 shown in FIGS. 3A and 3B with selective receive apertures applied. With reference to FIG. 18A, the ultrasound transducer probe 68 of FIGS. 3A and 3B was tested in silico for performance in imaging a midline and a 5 mm thick (3 cc volume) intraparenchymal hemorrhage (IPH). FIG. 18B shows an image of midline and IPH using the central transmit/receive pad C6 to generate an ultrasound imaging pulse at 800 kHz. FIG. 18C shows an image of midline and IPH using the side transmit/receive pad C1 at 800 kHz.

Further testing of the ultrasound transducer probe 68 was carried out by simulating the probe's performance while imaging a subdural hematoma (SDH). FIG. 19 shows the simulation medium for a 17 mm SDH beneath the skull layer.

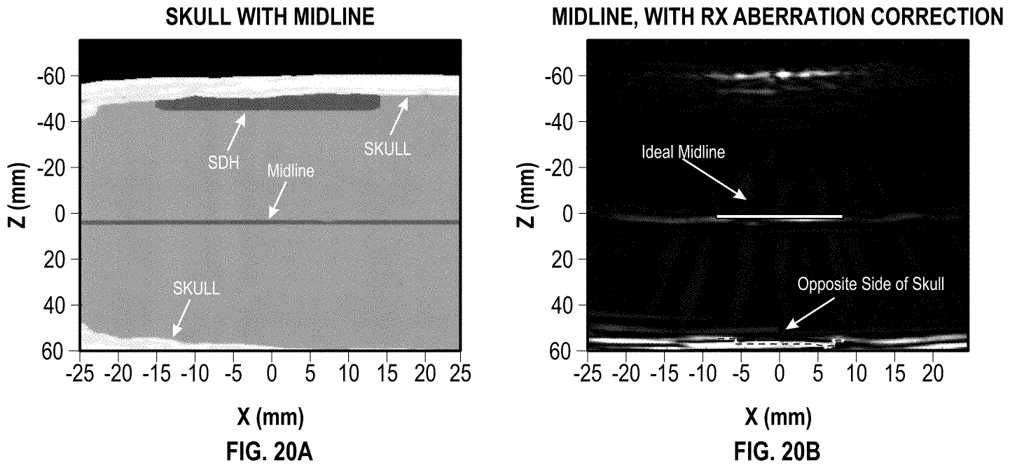

FIG. 20A shows a skull and a brain with a midline used in the simulation testing. FIG. 20B shows a reconstructed image from the ultrasound transducer probe 68 with skull aberration correction to the receive beamforming implemented. The correction shifts the midline to the expected position.

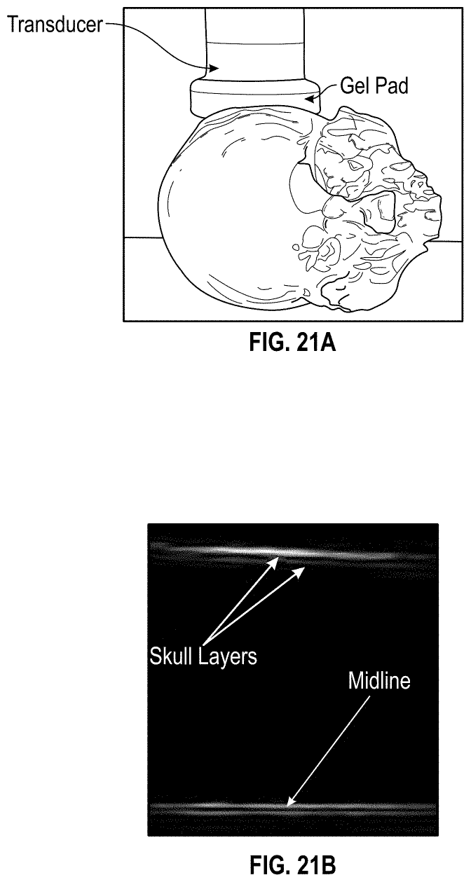

FIG. 21A shows a skull phantom filled with a brain phantom composed of agar that was used for testing the performance of the fabricated ultrasound transducer probe 68. The ultrasound image of the midline channel within the agar can be seen in FIG. 21B. Ultrasound imaging of another TBI phantom with a hematoma is shown in FIG. 21C.

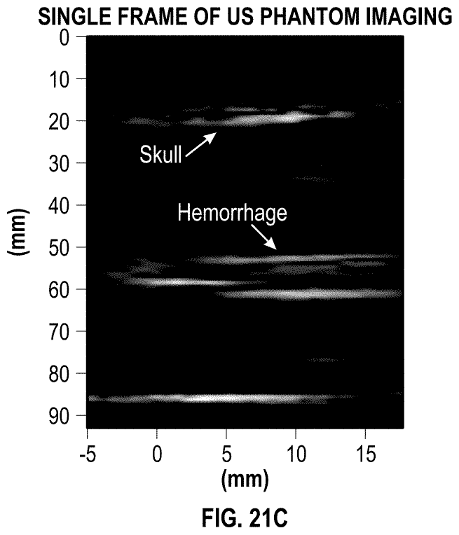

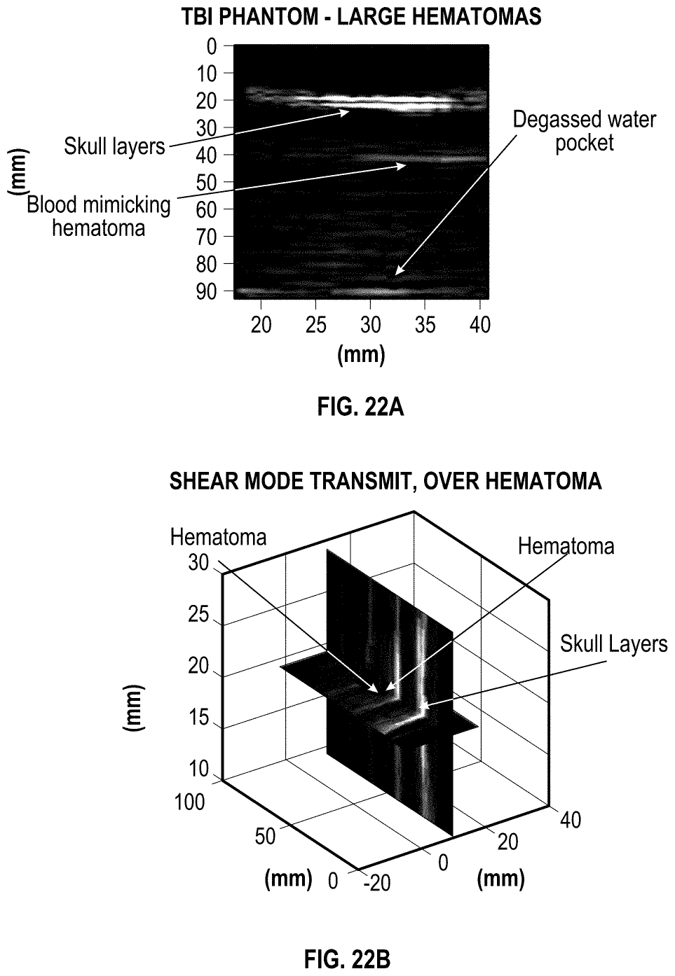

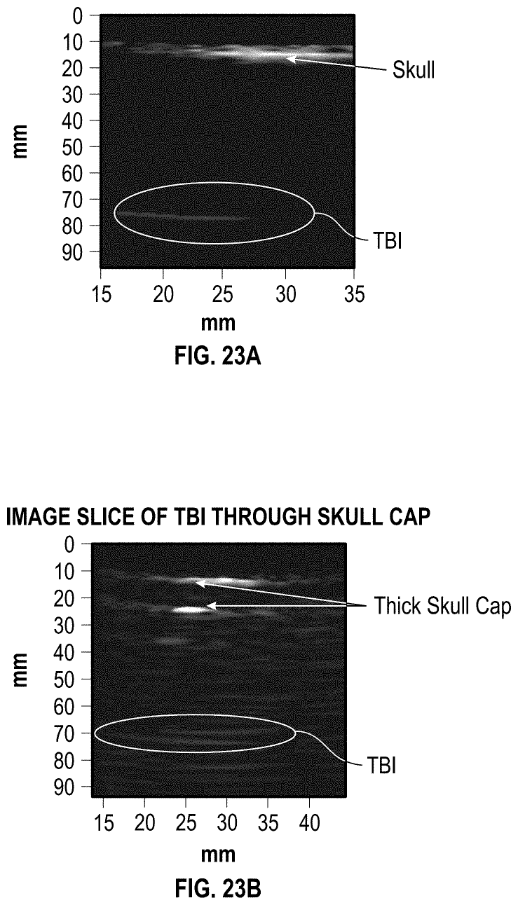

FIG. 22A shows a slice of ultrasound volume, showing the skull layers, a large hematoma, and a midline channel. FIG. 22B shows a cross section plot showing the skull layers and hematoma imaged with shear angle transmit ultrasound. Similarly, FIGS. 23A and 23B show imaged hematomas through a thick skull cap.

The dual wave ultrasound imaging system 50 utilizes a reflection mode imaging method, where the backscattered plane wave, transmitted by 128 transmit elements, is recorded by the larger array and beam formed onto a 3D grid to produce a 3D imaging data set. A post-processing algorithm enhances the contrast as a function of imaging depth and applies a top hat filter to sharpen the image. Compound imaging can also be performed, e.g., by adding additional transmission events with different steering angles (incident angles) to the frame. The 3D data set can be built from a synthetic receive aperture:

$$I ( l , m , n ) = {\sum }_{T = 1}^{N} {\int }_{t - w}^{t + w} \vert H \{ {\sum }_{R ∈ A} r ( t + Δ {t}_{T} , T , R ) \} \vert ,$$

where I is the voxel intensity for locations l, m and n, T is the transmit event, w is the window which describes the voxel size, t is time, r is the digitized received RF signal at element R, Δtᵣ is the beamforming time delay between element R and voxel (l, m, n) and A is the receive aperture, which controls which elements R will contribute to the image. The receive aperture may be adjusted to control which elements can contribute to each voxel. The receive aperture may be adjusted based on the receive incident angle, or the receive pad of the transducer. The beamforming delay may be adjusted to account for the bone layer. The algorithm may be parallelized for speed, and may be computed in part on a GPU processor. The bone layers and features below can be visualized slice-by-slice as a 2D contrast image, and as a 3D isometric surface plot, where the feature is located in the 2D slice image, and from that pixel value, an isometric surface plot may be generated. The software may selectively apply phase and amplitude correction to digitized receive RF signals, depending on the transmit and/or receive incident angle in order to correct for the distortion of longitudinal waves passing through the skull layer. The imaging algorithms and software may include methods to automatically detect the location of the top and bottom surfaces of the skull from the raw digitized received RF signals from both longitudinal waves transmitted through the skull, and from shear converted waves. An estimate of the phase shift and amplitude correction to apply to each receive element of the transducer can then be either calculated from the digitized received RF signals or calculated through simulation of transmission through the skull to account for variation in skull morphology over the region of interest. This element-wise phase shift correction can be included in reconstruction beamforming selectively to account for distortion of the bone. Filtering may be employed to the digitized received RF signals to remove multiple reflections between the bone and transducer surfaces. The imaging algorithms and software may include an algorithm to find multiple reflections from the skull, and then apply a filter to remove them from the digitized received RF signals. The imaging algorithms and software may employ contrast enhancement algorithms to selectively enhance the contrast of weak echoes within the brain. Post processing filters such as edge detection or sharpening filters may be employed. The images may be displayed as a 2D ultrasound image, as a series of 2D slices allowing the operator to scroll through the slices captured for one frame of imaging data or as a larger montage view, where the software takes the captured images from many positions, along with the positional data to co-register the ultrasound voxels (or pixels) and interpolate a larger montage image. In some embodiments, feature tracking 255 may be employed to enhance, improve or implement the co-registration of ultrasound pixels or voxels as the transducer is moved across the patient.

During development, the dual wave ultrasound imaging system 50 was tested with skull and brain phantoms within a water tank. A comparison of the ultrasound imaging to CT imaging of the phantoms was carried out.

Skull and TBI phantoms (FIG. 21A) were constructed from a full skull printed in Accura™ ClearVue™ plastic available from 3D Systems, which has acoustic properties similar to bone, and cadaver skull cap samples. Each skull cap was mounted to a Plexiglas plate, with fiduciary markers to facilitate co-registration of imaging modalities. The skull caps were rinsed in de-ionized water and degassed for over 3 hours. A 2% by weight agar solution was prepared using degassed de-ionized water to simulate tissue. An agar insert with a grid of ball bearings (e.g., 4 mm ball bearings) suspended inside was inserted into the agar to mimic shrapnel. To mimic hemorrhages, early phantoms utilized a balloon filled with blood-mimicking fluid or a flat pancake-shaped reservoir constructed from 3 mil (or 0.076 mm) low density polyethylene and filled with blood-mimicking fluid and gadolinium at a concentration of 20 mg/ml in order to enhance CT contrast. The TBI phantoms were imaged with the Toshiba Aquilion One CT imaging system in order to confirm the ultrasound imaging.

FIGS. 21A-21C show a TBI phantom fabricated from the printed skull. Imaging of a midline channel filled with degassed water is also shown (FIG. 21B). FIG. 21C shows an ultrasound imaging of a TBI hemorrhage phantom. The hemorrhage embedded in the agar is clearly visible. To avoid air pockets within the midline channel, the imaging took place in a tank full of degassed water. Typically, imaging of phantoms made from the printed skull are conducted in air, with a gel pad and ultrasound gel as coupling. Example images of blood mimicking hematoma from these phantoms as shown in FIGS. 21A-21C and 22A-22B. The TBI phantoms fabricated from skull cap samples were imaged inside a shallow bath of degassed water to keep the skull bone degassed. The gel pad is not excluded in this case, in order to ensure correct standoff, and to test imaging with the gel pad. FIGS. 23A-23B show imaging of the top surface of a blood filled pocket below a thick skull cap.

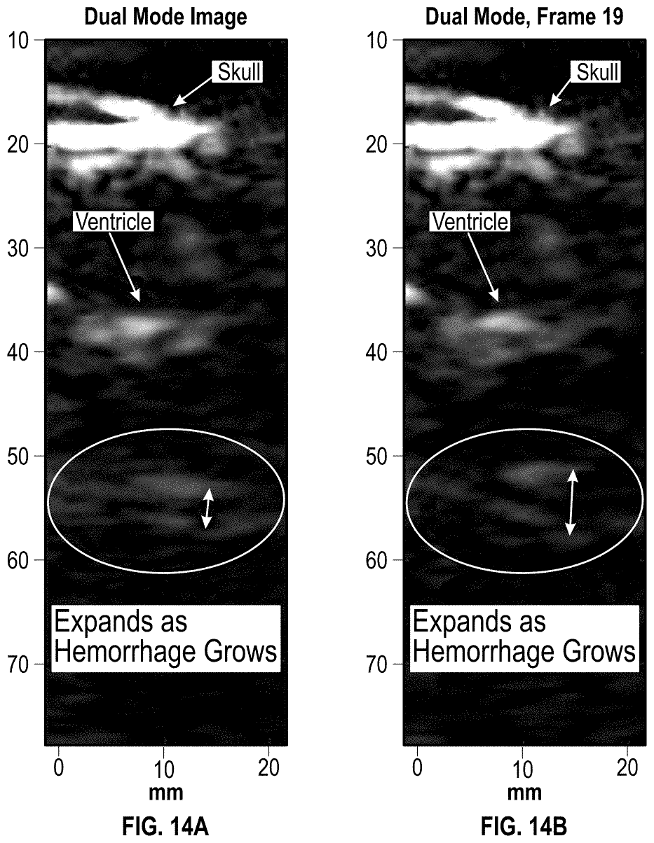

FIGS. 14A and 14B show the results of dual wave imaging mode of a commercially available realistic ultrasound brain phantom, with ventricles and progressively expanding simulated hemorrhage below a skull cap sample.

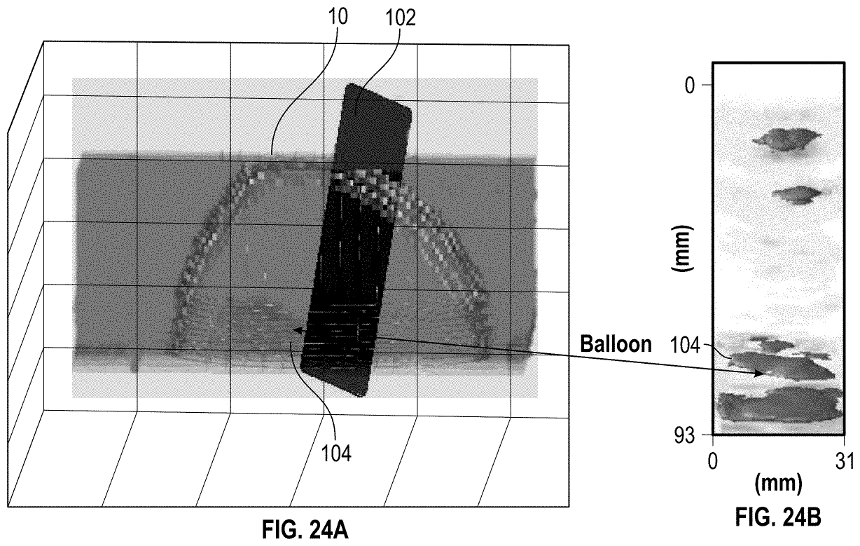

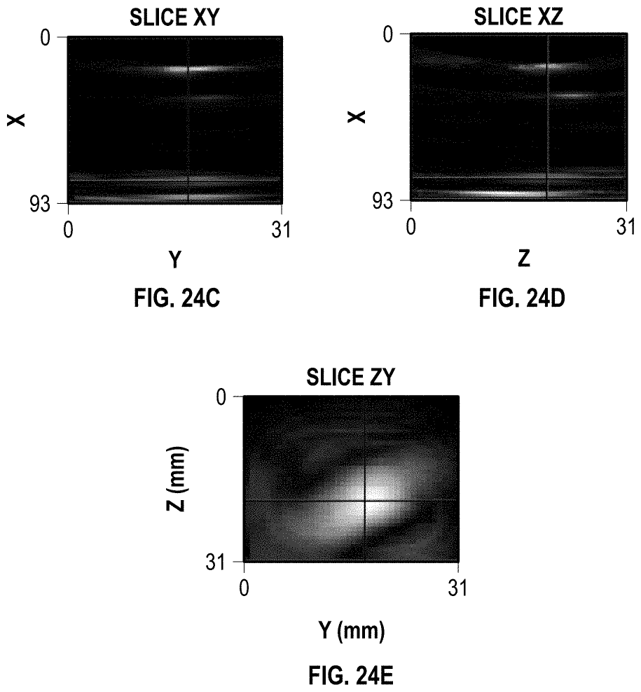

In order to validate the performance of the dual wave ultrasound imaging system 50 of the present invention, the scans produced by the dual wave ultrasound imaging system 50 were compared to CT scans. Fiduciary markers and anatomical features on the phantom skull were used to co-register the ultrasound and CT images to allow verification of imaging. FIGS. 24A-24E illustrate the field of view for a central slice 102 produced by the ultrasound transducer probe 68, for one pose, relative to the CT scan of a hemorrhage phantom 104. A 3D model of the pixel values show the blood-mimicking filled balloon below the skull cap 10. Cross sectional 2D images are also shown. The TBI phantom of a pancake shaped balloon and seven mm diameter tube filled with blood mimicking fluid encased in agar is imaged with the dual wave ultrasound imaging system 50. The location of the central slice 102 of ultrasound data is shown with respect to the phantom 104 in FIG. 24A. A 3D rendering of the ultrasound imaging volume, showing the tube and skull layers is shown in FIG. 24B. The ultrasound cross sectional views of the 3D imaging data for one frame of data is shown in FIGS. 24C-24E.

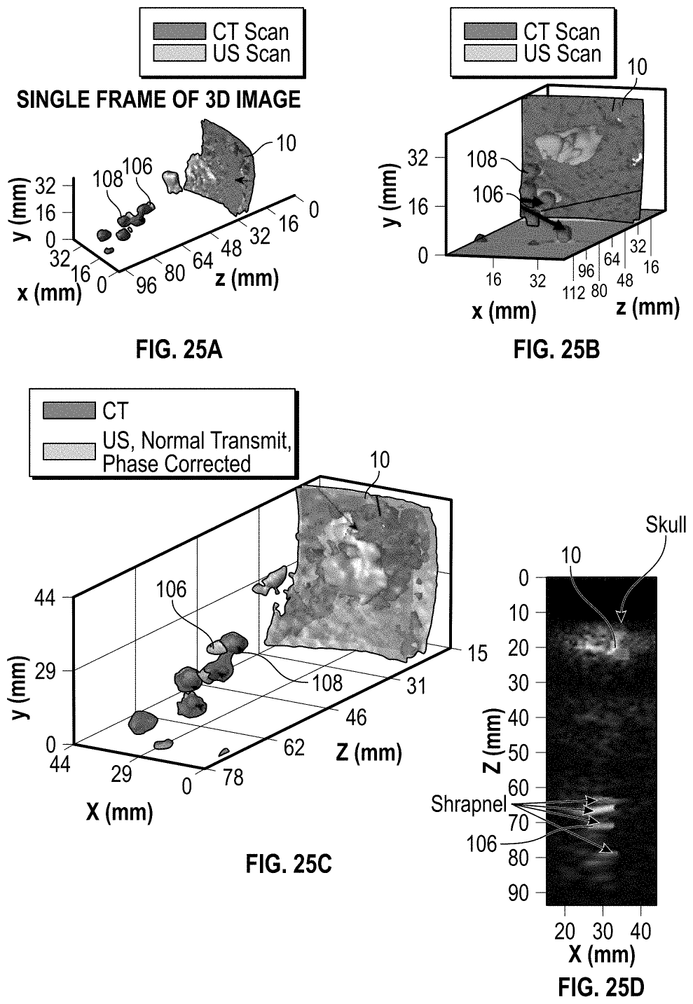

A comparison of the CT data for a shrapnel phantom to the single pose FOV of the transducer is shown in FIGS. 25A-25D. In FIGS. 25A-25D, the 3D isosurface rendering of the CT and ultrasound images shows the skull layers with embedded shrapnel beneath for 2 different transducer locations. Shrapnel 108 from the CT scan is shown in dark gray, and shrapnel 106 from the ultrasound scan is shown in light gray. Some reverberation artifacts can be seen below the skull layer. The ball bearing shrapnel locations are confirmed with the CT scan. The images in FIGS. 25A and 25B show the shrapnel imaged below the scan for one embodiment of the image reconstruction algorithm. FIGS. 25C and 25D show the image quality in another embodiment, where phase correction and skull reflection filtering algorithms were employed.

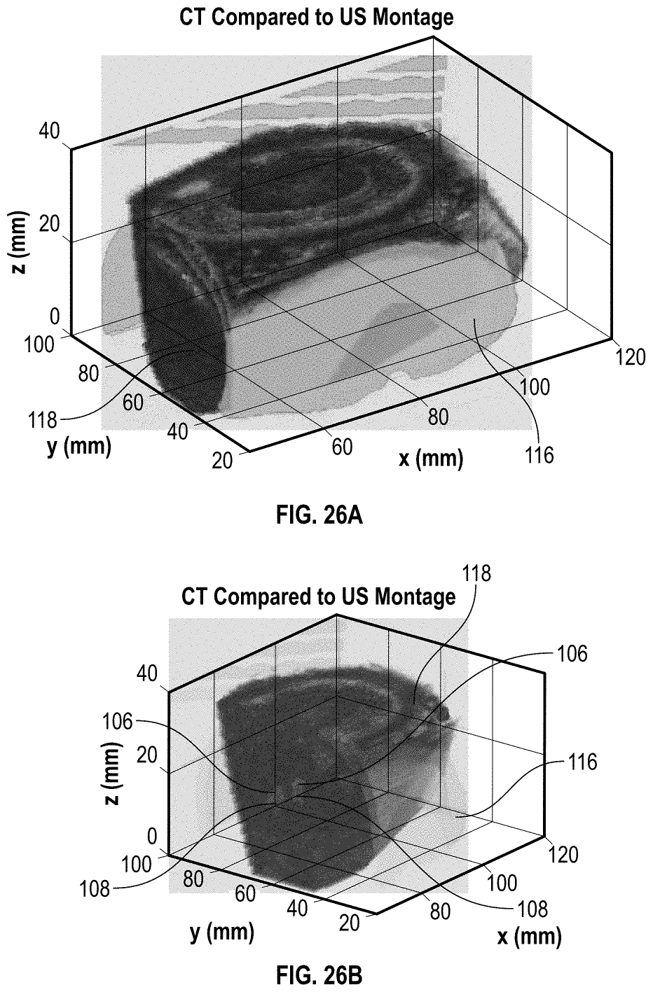

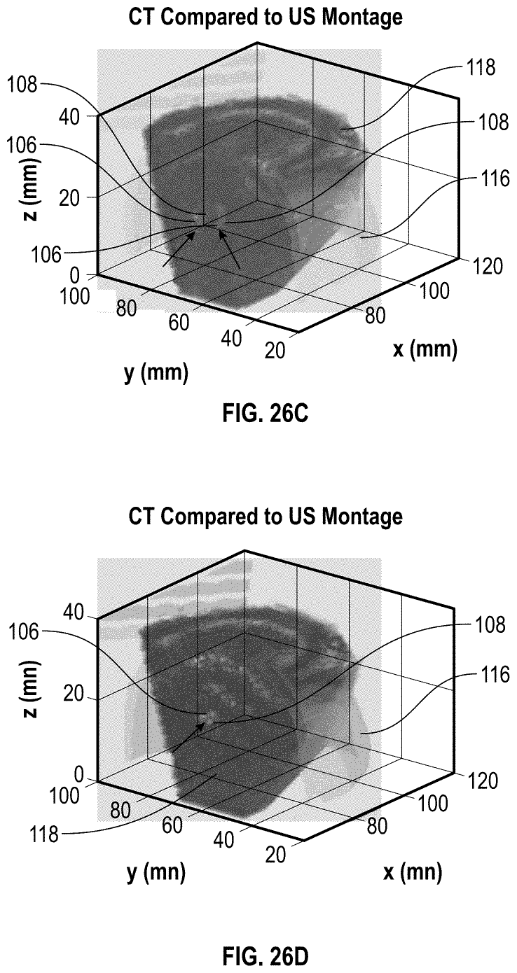

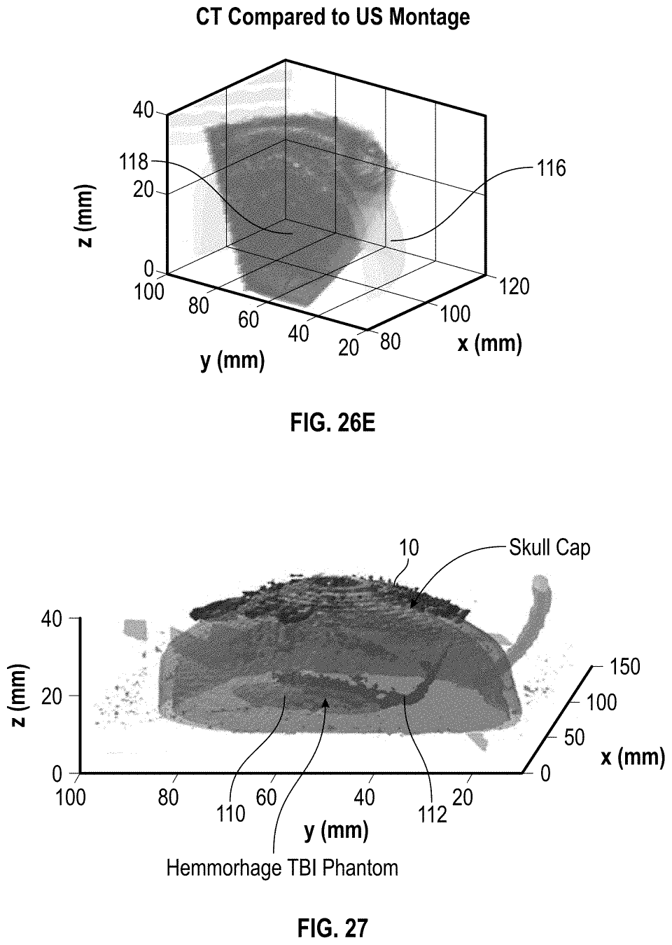

The montage whole brain imaging functionality of the dual wave ultrasound imaging system 50 is shown in FIGS. 26A-26E. The image created from 200 ultrasound imaging frames, or poses, which have been co-registered using position tracking data, and interpolated to create a 3D ultrasound montage which is overlaid on top of a CT isosurface 116 to verify the shrapnel location 108. The CT surface plots of the skull cap 116 and ball bearing shrapnel 108 is shown. Each figure shows a progressive cross section view of the 3D ultrasound volume 118, revealing the ball bearings 106 (ultrasound scan shrapnel) and 108 (CT scan shrapnel) within the phantom.

FIG. 27 shows the isosurface of a simulated blood pocket below the skull 10. The simulated blood pocket 110 is identified by the CT scan. The isosurface rendering of the ultrasound 3D montage is shown, where the blood pocket 112 is clearly visible.

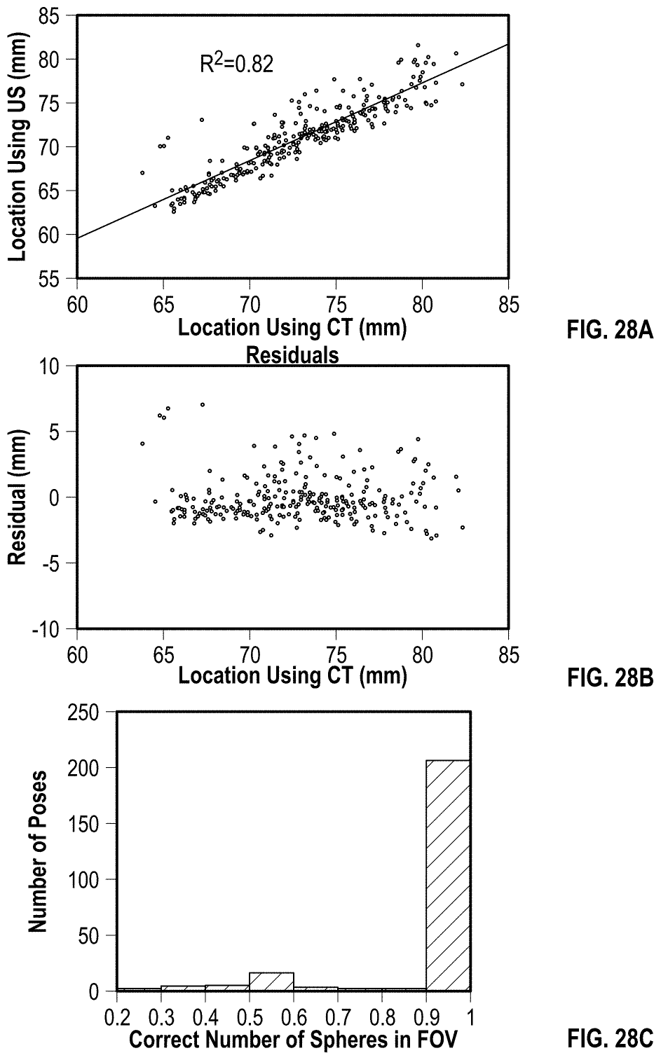

FIGS. 28A-28C show analysis of a ball bearing phantom by comparing the ultrasound scan to the CT scan. The visibility of the shrapnel is illustrated while the ultrasound transducer probe 68 is allowed to move freely. FIG. 28A shows over 500 frames of imaging data that were inspected for the ball bearing feature. FIG. 28A shows the percentage of spheres visible in each frame of the ultrasound scan for which they appear in the CT data. Registration error between the CT and ultrasound data sets is shown. The plot in FIG. 28C shows the agreement between the location of the ball bearing in the ultrasound scan and the CT scan. Residuals of the linear regression of the location data of the ball bearing is shown in FIG. 28B.

As discussed herein, the nature of the propagation of longitudinal ultrasound waves through a bone layer depends on the transmission angle of the longitudinal ultrasound waves. Likewise, the nature of waves reflected from a target depends on the receiving angle as would be appreciated by one of ordinary skill in the art having the benefit of this disclosure. FIG. 29 is a schematic showing a transmission configuration 300 for the transmission of longitudinal ultrasound waves 301 a first angle such that there is zero wave conversion as the waves propagate from the transducer 68 through tissue 310, a bone layer 320, tissue 330, and strike a target 340. Likewise, there is zero wave conversion as the waves reflect back to the transducer 68 as longitudinal waves. The first angle may be 0 degrees to ˜25 degrees. Transducer 68 transmits longitudinal ultrasound waves 301A that pass through tissue 310. The waves 301B propagate through the bone layer 320 as longitudinal waves and exit the bone layer 320 and the waves 301C propagate through tissue 330 as longitudinal waves. Upon striking the target 340, the waves 302A are reflected back towards the transducer 68 and propagate through the tissue 330 as longitudinal waves. The reflected waves 302A which are incident upon the bone layer 320 at an angle less than 25 degrees will propagate through bone layer 320 as longitudinal waves 302B. The reflected waves 302C then travel through tissue 310 as longitudinal waves and are received by the transducer 68.

FIG. 30 is a schematic showing a transmission configuration 400 for the transmission of longitudinal ultrasound waves at a second angle such that there is a double wave conversion as the waves propagate from the transducer 68 through tissue 410, bone layer 420, tissue 430, and strike target 440. The second angle may be 25 degrees-60 degrees. Transducer 68 transmits longitudinal ultrasound waves 401A that pass through tissue 410. The waves convert to shear waves 401B as they propagate into the bone layer 420 due to the transmission at the second angle. As the shear waves 401B exit the bone layer 420, the waves convert back to longitudinal waves 401C as they propagate through tissue 430. Upon striking the target 440, the waves 402A are reflected back towards the transducer 68 and propagate through the tissue 430 as longitudinal waves. The reflected waves 402A which are incident upon the bone layer 420 at an angle less than 25 degrees will propagate through bone layer 420 as longitudinal waves and reflected waves 402B which will then be transmitted into tissue 410 as longitudinal waves 402C, which are received by the transducer 68.

FIG. 31 is a schematic showing a transmission configuration 500 for the transmission of longitudinal ultrasound waves a third angle such that there is no wave conversion as the transmitted wave propagates through the bone layer 520 and a double wave conversion as the reflected waves propagate from the target 540 back to the transducer 68 through tissue 510, bone layer 520, and tissue 530. The transmission at the third angle causes zero wave conversion as the waves propagate from the transducer 68 through the tissue 510, bone layer 520, and tissue 530 to the target 540. The third angle may be the same as the first angle. For example, the third angle may be 0 degrees to 25 degrees like the first angle discussed in regard to FIG. 29. However, the waves of FIG. 31 differ from the waves of FIG. 29 due to a different receive angle than the receive angle associated the first angle of FIG. 29. Transducer 68 transmits longitudinal ultrasound waves 501A that pass through tissue 510. The waves 501B propagate through the bone layer 520 as longitudinal waves due to the transmission at the third angle. The waves 501C continue to propagate as longitudinal waves through tissue 530. Upon striking the target 540, the waves 502A are reflected back towards the transducer 68 and propagate through the tissue 530 as longitudinal waves. The reflected waves 502A which are incident upon the bone layer at an angle between ˜25 degrees and 60 degrees will be converted in part (incident angle <≃30 degrees) or in total (incident angle >30 degrees) to shear waves 502B, and propagate through the bone layer 520. As the shear waves 502B exit the bone layer 520, the reflected waves 402C convert back to longitudinal waves 502C and then travel through tissue 510 as longitudinal waves, which are received by the transducer 68.

FIG. 32 is a schematic showing a transmission configuration 600 for the transmission of longitudinal ultrasound waves a fourth angle such that there is a quadruple wave conversion as the waves propagate from the transducer 68 through tissue 610, bone layer 620, tissue 630, and strike target 640 and are reflected back to the transducer 68. The fourth angle may be the same as the second angle discussed in regard to FIG. 30. For example, the fourth angle may be 25 degrees to 60 degrees like the second angle of FIG. 30. However, the waves of FIG. 32 differ from the waves of FIG. 30 due to a different receive angle than the receive angle associated the second angle of FIG. 30. Transducer 68 transmits longitudinal ultrasound waves 601A that pass through tissue 610. The waves convert to shear waves 601B due to the transmission at the fourth angle and propagate through the bone layer 620 due to the transmission at the fourth angle. As the shear waves 601B exit the bone layer 620, the waves 601C convert back to longitudinal waves 601C as they propagate into tissue 630. Upon striking the target 640, the waves 602A are reflected back towards the transducer 68 and propagate through the tissue 630 as longitudinal waves. The reflected waves 602A which are incident upon the bone layer 620 at an angle between ˜25 degrees and 60 degrees will be converted in part (incident angle <≃30 degrees) or in total (incident angle >≃30 degrees) to shear waves 602B. As the shear waves 602B exit the bone layer 620, the reflected waves 602C convert back to longitudinal waves 602C and then travel through tissue 610 as longitudinal waves, which are received by the transducer 68.

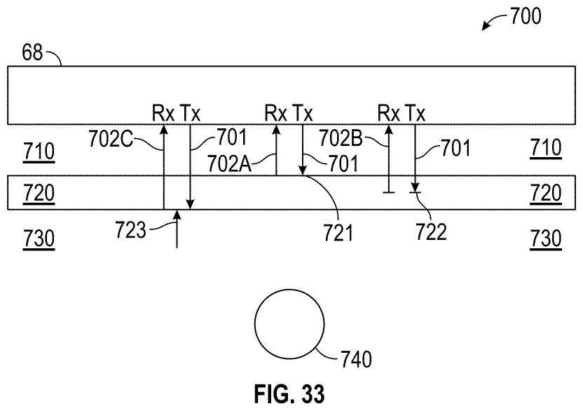

FIG. 33 is a schematic showing a transmission configuration 700 for the transmission of longitudinal ultrasound waves at a fifth angle such that a portion are reflected back off the bone surface, or trabecular bone, or the interior surface of the bone, to be received by the transducer as longitudinal waves 702. The fifth angle may be [0 to 60 degrees]. Transducer 68 transmits longitudinal ultrasound waves 701 that upon striking the exterior surface of the bone 721 a portion of the ultrasound waves reflect back to the transducer as longitudinal waves 702A; or, upon striking the trabecular bone 722 a portion of the ultrasound waves reflect back to the transducer as longitudinal waves 702B; or, upon striking the interior surface of the bone 723 a portion reflect back to the transducer as longitudinal waves 702C.

### CONCLUSION

The dual wave ultrasound imaging 50 of the type described herein may be used in a wide variety of trans-bone imaging and non-imaging applications, include imaging brain structures below skull bone 10, such as, without limitation, the ventricles, pathological conditions affecting the brain such as hemorrhage, hydrocephalus, intracranial pressure (ICP), foreign bodies, and other conditions, in order to detect and/or assist in diagnosis and ongoing monitoring of traumatic brain injury, stroke, tumors, etc., and may be used in a wide variety other of trans-bone imaging and non-imaging applications, including without limitation: other brain and intracranial diagnosis and monitoring, sinus opacification diagnosis and other ear-nose-throat (ENT) diagnosis and treatment, and intraoperative surgical imaging and navigation, etc. The ultrasound imaging algorithms and software implemented by the host controller 52 with an enhanced frame rate enables tomographical image reconstruction of whole or partial brain imaging of dynamic features of the target subject/region including but not limited to blood flow, brain shift, effusions, hemorrhages, etc.

Although the above discussion discloses various exemplary embodiments of the invention, it should be apparent that those skilled in the art can make various modifications that will achieve some of the advantages of the invention without departing from the true scope of the invention. Any references to the “invention” are intended to refer to exemplary embodiments of the invention and should not be construed to refer to all embodiments of the invention unless the context otherwise requires. The described embodiments are to be considered in all respects only as illustrative and not restrictive.

Thus, certain exemplary embodiments can provide 2D imaging, 3D imaging and tomography, and 4D time lapse imaging and tomography (i.e., 3D tomography in real-time) of whole or partial brain images through the skull 10, or through other bones and structures such as the sternum (e.g., for imaging of the heart or esophagus), ribs, hip, pelvis, etc. via ultrasound. The dual wave ultrasound imaging system 50 can provide live 3D images from within the field of view of the ultrasound transducer probe 68 as it is held in a single position. That limited live field of 3D view can be expanded to include post-processing views of the entire brain via interpolation and co-registration of ultrasound pixels (or voxels) to a global co-ordinate system from multiple fields of view by tracking free manual movement of the ultrasound transducer probe 68 by tracking system 84. The compilation of imaging data can be processed post-scan to produce cross-sectional (orthogonal) images of the brain 244 (i.e., computed tomography of the brain 252) via non-invasive, dual wave imaging mode ultrasound).

We claim:

1. An ultrasound imaging system comprising:
an ultrasound transducer probe includes a face configured to contact a subject, the face including an array of transducer elements, the array of transducer elements includes at least one first transmit pad that includes at least one first active transducer element, at least one second transmit pad that includes at least one second active transducer element, and at least one receive pad, wherein the at least one first active transducer element is capable of transmitting longitudinal ultrasound waves at a first incident angle with respect to a bone of the subject so that waves may propagate through the bone as shear waves and wherein the at least one second active transducer element is capable of transmitting longitudinal ultrasound waves at a second incident angle with respect to the bone so that the waves may propagate through the bone as longitudinal waves;
a host controller;
an ultrasound driving system;
an ultrasound transducer probe;
an ultrasound system switch that connects the ultrasound driving system to the ultrasound transducer probe, wherein the host controller controls operation of the ultrasound transducer probe via the ultrasound driving system;
wherein the host controller commands the ultrasound driving system to generate radio frequency (RF) signals that are used by the transducer probe to generate ultrasound waves;
wherein upon receipt of commands from host controller, the ultrasound driving system causes the ultrasound transducer probe to generate ultrasound waves at the first incident angle and at the second incident angle;
wherein the ultrasound driving system captures electronic signals produced by ultrasound waves received by the at least one receive pad of the ultrasound transducer probe via the ultrasound system switch and digitizes the received electronic signals; and
wherein the host controller forms an image of the subject based on the digitized received electronic signals.

2. The ultrasound imaging system of claim 1, wherein the first incident angle is above a critical angle for longitudinal waves and below a critical angle for shear waves and wherein the second incident angle is below the critical angle for longitudinal waves.

3. The ultrasound imaging system of claim 1, wherein the least one first transmit pad is configured to receive ultrasound waves.

4. The ultrasound imaging system of claim 3, the at least one first transmit pad further comprises a first centrally located pad and an additional pad offset from the first centrally located pad, wherein the first central located pad and the additional pad may be configured to transmit, receive, or both.

5. The ultrasound imaging system of claim 1, comprising a gel pad or gel positioned between the ultrasound transducer and the subject.

6. The ultrasound imaging system of claim 1, wherein the bone of the subject is the head.

7. The ultrasound imaging system of claim 1, wherein the image formed by the host controller is comprised of pixels or voxels.

8. The ultrasound imaging system of claim 8, comprising a position tracking system coupled to the host controller, wherein the host controller co-registers the pixels or voxels of the image with a global coordinate system based on tracking information from the position tracking system.

9. The ultrasound imaging system of claim 9, wherein the host controller interpolates the pixels or voxels co-registered with the global coordinate system to form a larger montage image.

10. The ultrasound imaging system of claim 1, wherein the ultrasound transducer probe having a plurality of channels with each channel of the plurality of channels corresponding to an individual transducer element of the array of transducer elements.

11. The ultrasound imaging system of claim 11, wherein the ultrasound system switch includes fuses to limit a maximum voltage to be applied to an individual transducer element of the array of transducer elements.

12. The ultrasound imaging system of claim 11, wherein the ultrasound system switch monitors the channels to determine a delivered ultrasound wave has completed within an allotted time.

13. The ultrasound imaging system of claim 11, wherein the ultrasound system switch is configured to rapidly switch between channels to allow a single channel to be used for transmit and receive.

14. The ultrasound imaging system of claim 14, wherein the ultrasound system is configured to switch from a first channel after delivering an RF signal, to a second channel before a reflected ultrasound wave is received from the delivered ultrasound wave from the transducer element of the first channel.

15. The ultrasound imaging system of claim 1, wherein the ultrasound system switch selectively couples the RF signals from the ultrasound driving system with the array of transducer elements.

16. The ultrasound imaging system of claim 1, the ultrasound system switch having a plurality of channels, wherein the channels correspond to a total number of transducer elements of the array of transducer elements, the ultrasound system switch comprising:
a plurality of interfaces, each interface of the plurality of interfaces configured for a connection with a different segment from among the array of transducer elements;
an interface configured to connect to the ultrasound driving system; and
wherein the ultrasound system switch is configured to limit the ways voltage may be applied to the array of transducer elements from the ultrasound driving system.

17. An ultrasound transducer probe comprising:
a face configured to contact a subject; and
an array of transducer elements including at least one first transmit pad and at least one second transmit pad, the first transmit pad includes at least one first active transducer element and the at least one second pad includes at least one second active transducer element, and at least one receive pad, wherein the at least one first active transducer element is capable of delivering longitudinal ultrasound waves at a first incident angle with respect to a bone of the subject that produces shear waves through the bone and wherein the at least one second active transducer element is capable of delivering longitudinal ultrasound waves at a second incident angle with respect to the bone of the subject so that it produces longitudinal waves though the bone.

18. The ultrasound transducer probe of claim 17, wherein the at least one receive pad has a first footprint and the at least one first transmit pad has a second footprint smaller than the first footprint.

19. The ultrasound transducer probe of claim 17, wherein the first active transducer element and the second active transducer element are rectangular in shape.

20. The ultrasound transducer probe of claim 17, wherein the first active transducer element and the second active transducer element are configured to transmit longitudinal ultrasound waves and are configured to receive reflected longitudinal ultrasound waves.

21. The ultrasound transducer probe of claim 17, wherein each of the transducer elements of the array of transducer elements is an active transducer element.

22. The ultrasound transducer probe of claim 17, wherein the at least one first pad is centrally located on the face and wherein the at least one second pad is offset from the at least one first pad, and wherein the second incident angle is below a shear critical angle.

23. The ultrasound transducer probe of claim 22, wherein the second incident angle is above a longitudinal critical angle.

24. The ultrasound transducer probe of claim 23, wherein the second incident angle is below a longitudinal critical angle.

25. The ultrasound transducer probe of claim 23, wherein the array of transducer elements are configured to receive quadruple conversion longitudinal ultrasound waves.

26. An ultrasound imaging method comprising:
transmitting longitudinal ultrasound waves via an ultrasound probe toward a target at a plurality of incident angles, wherein at least a first incident angle is below a longitudinal wave critical angle and wherein at a second incident angles is above the longitudinal wave critical angle and below a shear wave critical angle;
receiving reflected longitudinal ultrasound waves via the ultrasound probe;
producing received radio frequency (RF) signals via the ultrasound probe based on the received reflected longitudinal ultrasound waves;
receiving backscattered longitudinal ultrasound waves via the ultrasound probe;
producing received RF signals via the ultrasound probe based on the received backscattered longitudinal ultrasound waves;
digitizing the received RF signals to form digitized RF signals; and
processing the digitized RF signals to form an image of the target.

27. The ultrasound imaging method of claim 26, wherein the target is soft tissue, and the incident angles are with respect to normal to the plane of a bone layer, and the longitudinal ultrasound waves are transmitted through the bone layer.

28. The ultrasound imaging method of claim 27, wherein first incident angle enables longitudinal waves to pass through the bone and wherein the second incident angle enables a quadruple conversion of the longitudinal waves within the bone.

29. The ultrasound imaging method of claim 27, wherein transmitting longitudinal ultrasound waves further comprises:
transmission of longitudinal ultrasound waves such that the longitudinal ultrasound waves propagate through the bone layer as longitudinal waves which then reflect and propagate back through the bone layer as longitudinal waves to be received by the transducer as reflected longitudinal waves;
transmission of longitudinal ultrasound waves such that the longitudinal ultrasound waves propagate through the bone layer as shear waves, which convert to longitudinal waves upon exiting the bone layer, which then reflect back at such angle that the reflected waves propagate through and exit the bone layer as longitudinal waves to be received by the transducer as longitudinal waves;
transmission of longitudinal ultrasound waves such that the longitudinal ultrasound waves propagate through the bone layer as longitudinal waves which then reflect back at such angle that the reflected waves propagate through the bone layer as shear waves, which then convert from shear waves to longitudinal waves upon exiting the bone layer to be received by the transducer as longitudinal waves; and
transmission of longitudinal ultrasound waves such that the longitudinal ultrasound waves propagate through the bone as shear waves, which then exit the bone layer and convert to longitudinal waves, which then reflect back at such an angle that they propagate back through the bone layer as shear waves, and then convert back again to longitudinal waves as they exit the bone layer to be received by the transducer as longitudinal waves.

30. The ultrasound imaging method of claim 29, further comprising utilizing any transmitted longitudinal ultrasound waves reflected off one or more of an exterior bone surface, a trabecular bone, or an interior bone surface to characterize a bone morphology and calculate phase shifts introduced to propagating ultrasound waves.

31. The ultrasound imaging method of claim 28, wherein digitizing the RF signals to form digitized ultrasound waves further comprises digitizing the RF signals from received quadruple conversion longitudinal waves.

32. The ultrasound imaging method of claim 26, comprising applying filtering to the digitized RF signals from the ultrasound waves to remove multiple reflections.

33. The ultrasound imaging method of claim 26, comprising correcting the digitized received RF signals from the ultrasound waves for phase shift.

34. The ultrasound imaging method of claim 26, comprising determining characteristics of the bone.

35. The ultrasound imaging method of claim 34, comprising correcting the digitized RF signals from the ultrasound waves based on the characteristics of the bone.

36. The ultrasound imaging method of claim 26, comprising estimating a phase shift of the ultrasound waves due to the bone.

37. The ultrasound imaging method of claim 36, comprising correcting the digitized received RF signals from the ultrasound waves based on the estimated phase shift.

38. The ultrasound imaging method of claim 26, utilizing a synthetic receive aperture to select the received RF signals that will contribute to the image.

39. The ultrasound imaging method of claim 38, further comprising correcting the digitized RF signals from the received ultrasound waves based on a synthetic receive aperture.

40. The ultrasound imaging method of claim 26 further comprising:
preprocessing the digitized RF signals to create pre-processed RF signals;
selecting data from the preprocessed RF signals to isolate selected RF signals;
reconstructing the image from the selected RF signals; and
post processing the image.

41. The ultrasound imaging method of claim 40, wherein preprocessing includes depth enhancement.

42. The ultrasound imaging method of claim 40, wherein preprocessing includes filtering bone matter reflection from the received RF signals.

43. The ultrasound imaging method of claim 40, wherein preprocessing includes characterizing bone matter.

44. The ultrasound imaging method of claim 40, wherein preprocessing includes estimating a phase shift introduced to the ultrasound waves by a bone layer.

45. The ultrasound imaging method of claim 40, wherein selecting data further comprises identifying and selecting a transmit pad, selecting incident angles from the plurality of incident angles, and selecting receive incident angles.

46. The ultrasound imaging method of claim 40, wherein reconstructing the image further comprises aberration correction to correct for a distortion introduced by a bone layer.

47. The ultrasound imaging method of claim 40, wherein reconstructing the image further comprises beamforming to a three-dimensional ultrasound grid.

48. The ultrasound imaging method of claim 40, wherein preprocessing further comprises contrast enhancement.

49. The ultrasound imaging method of claim 40, wherein preprocessing further comprises using image enhancement filters.

50. The ultrasound imaging method of claim 40, wherein the image comprises pixels or voxels.

51. The ultrasound imaging method of claim 50, wherein preprocessing further comprises co-registering the pixels or voxels to a global coordinate system.

52. The ultrasound imaging method of claim 51, wherein co-registering the pixels or voxels utilizes one or more of optical tracking, magnetic tracking, kinetic tracking, or software-based feature tracking.

53. The ultrasound imaging method of claim 40, wherein preprocessing further comprises creating a three-dimensional interpolated montage of bone and soft tissue.

54. The ultrasound imaging method of claim 40, wherein preprocessing further comprises creating a two-dimensional slice from one field of view.

55. The ultrasound imaging method of claim 40, wherein preprocessing further comprises creating three-dimensional orthogonal slices of ultrasound images from one field of view.

56. The ultrasound imaging method of claim 40, wherein preprocessing further comprises creating a whole soft tissue two-dimensional slice.

57. The ultrasound imaging method of claim 40, wherein preprocessing further comprises creating three-dimensional tomography images from one field of view.

58. The ultrasound imaging method of claim 40, wherein preprocessing further comprises creating whole soft tissue three-dimensional orthogonal slices.

59. The ultrasound imaging method of claim 40, wherein preprocessing further comprises creating a four-dimensional visualization of a soft tissue.

60. The ultrasound imaging method of claim 40, further comprising using a synthetic receive aperture to determine how the plurality of incidence angles will affect reconstructing the image.

61. The ultrasound imaging method of claim 60, further comprising using the synthetic receive aperture to determine how receive angles of incidence will affect reconstructing the image.
